SEQUENCE LISTING (5 sequences)

SEQ ID NO: 1 - protein, 26 aa, H. sapien
TSISYGDEYPDYLDSIVVLEDLSPLC

SEQ ID NO: 2 - protein, 10 aa, H. sapien
FAFSKALPRS

SEQ ID NO: 3 - protein, 14 aa, H. sapien
FPGQFAFSKALPRS

SEQ ID NO: 4 - protein, 26 aa, Mus musculus
DSGIYDDEYSDGFGYFVDLEEASPWC

SEQ ID NO: 5 - protein, 163 aa, H. sapien
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

(19) United States
(12) Patent Application Publication
(10) Pub. No.: US 2005/0202029 A1
(43) Pub. Date: Sep. 15, 2005

(54) Family of cystatin-related chemoattractant proteins

(75) Inventors: Brian A. Zabel, Mountain View, CA (US); Eugene C. Butcher, Portola Valley, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Board of Trustees of the Leland Stanford Junior University

(21) Appl. No.: 10/958,527

(22) Filed: Oct. 4, 2004

Related U.S. Application Data
(60) Provisional application No. 60/508,360, filed on Oct. 3, 2003.

(51) Int. Cl. A61K 38/17; A61K 39/00

(52) U.S. Cl. 424/184.1; 514/12

(57) ABSTRACT

Methods are provided to modulate the trafficking of leukocytes through interactions with one or more of a class of chemoattractant proteins having a cystatin-like structure. Exemplary of proteins in this class is chemerin, which interacts with the receptor CMKLR1. The chemoattractant polypeptide, or agonists of the chemoattractant receptor, act to concentrate responding leukocytes at a site of interest. Agonists and antagonists of the chemoattractant modulate immune responsiveness.

Chemokines are a superfamily of small, secreted, cytokines that are involved in a variety of immune and inflammatory responses, acting primarily as chemoattractants and activators of specific types of leukocytes. Some members of this family were initially identified on the basis of their biological activities, others were discovered using subtractive hybridization or signal sequence trap cloning strategies. Known chemokines exhibit from 20% to over 90% identity in their predicted amino acid sequences. Chemokines mediate their activities by binding to target cell surface chemokine receptors, many of belong to the large family of G protein-coupled, seven transmembrane (7 TM) domain receptors. Leukocytes have generally been found to express more than one receptor type, and the various receptors are known to exhibit overlapping ligand specificities.

Three classes of chemokines were originally defined, based on the arrangement of the conserved cysteine residues in the mature proteins: the CXC or α chemokines have one amino acid residue separating the first two conserved cysteine residues; the CC or β chemokines have adjacent first conserved cysteine residues; the C or γ chemokines lack two of the four conserved cysteine residues.

A more recent classification uses physiological features, which include conditions and locations of chemokine production as well as cellular distribution of chemokine receptors, to distinguish between “inflammatory” (or inducible) chemokines and “homeostatic” (or constitutive) chemokines. Inflammatory chemokines are expressed in inflamed tissues by resident and infiltrated cells on stimulation by pro-inflammatory cytokines, or during contact with pathogenic agents. This group of chemokines is specialized for the recruitment of effector cells, including monocytes, granulocytes and effector T cells.

Homeostatic chemokines are produced in discrete microenvironments within lymphoid or non-lymphoid tissues such as the skin and mucosa. These constitutively produced chemokines are involved in maintaining physiological traffic and positioning of cells that mainly belong to the adoptive immune system during hematopoiesis, antigen sampling in secondary lymphoid tissue and immune surveillance.

Chemokines and their receptors help control the specificity of lymphocytes, including memory cells. For example, it has been shown that chemokines expressed by epithelial cells can selectively recruit T cells and/or B cells into skin, or the gut. The differential expression of particular chemokines within epithelial tissues suggests that organ systems previously thought to be relatively immunologically uniform may have important differences in terms of their immune character, while those thought to be more diverse may be linked in a previously unrecognized way.

Chemokines and their receptors are also important in dendritic cell maturation. For example, Fushimi (2000) J. Clin. Invest. 105(10):1383-93 explores the use of MIP-3α on the local accumulation of dendritic cells and anti-tumor immunity; and Vicari et al. (2000) J. Immunol. 165:1992 test the antitumor effects of the mouse chemokine 6Ckine/SLC. In addition to chemokines, cytokines may modulate the migration of dendritic cells (Wang et al. (1999) J. Leuk. Biol. 66(1):33-9), including interleukin (IL)-1, tumor necrosis factor alpha, and IL-10. Both the presence of the chemokines and cytokines, and the expression of the appropriate receptors may be involved. (Mantovani et al. (1998) Eur. Cytokine Network 9(3 Suppl):76-80).

Plasmacytoid dendritic cells (pDC) represent a small (<0.5%) but versatile subset of circulating leukocytes functioning at the interface between adaptive and innate immunity. Current evidence suggests that pDCs may share migratory properties with naïve T cells, expressing a combination of homing and chemokine receptors (L-selectin, CXCR4, CCR7 and α4 integrins) capable of facilitating traffic between blood and secondary lymphoid tissues, and responding to homeostatic chemokines, either CXCL12 in the immature state, or CCR7 ligands following in vitro maturation. pDC are present in diverse tissue sites however, often associated with the inflammation and lymphocyte infiltrates, and have been reported in reactive tonsils, inflamed nasal mucosa, thymus, cutaneous lesions (herpes zoster, skin blisters simulating syphilic infection (triggered by lipopeptide analogues of Treponema pallidum), psoriasis vulgaris, lupus erythematosus, contact dermatitis, but not atopic dermatitis, melanoma), peritoneal lavage fluid, and ovarian epithelial tumor. The mechanisms by which pDCs traffic from the blood to extralymphoid tissue sites is not well understood. Since blood pDCs do not respond to inflammatory chemokines, additional chemoattractant regulators may be involved.

The manipulation of leukocyte location in the body, particularly in combination with the use of the cells in immune responsiveness, is of great interest for its potential to provide for improved methods of immunization and for modulation of immune responses. The present invention addresses these methods.

Relevant Literature

The role of chemokines in leukocyte trafficking is reviewed by Baggiolini (1998) Nature 392:565-8, in which it is suggested that migration responses in the complicated trafficking of lymphocytes of different types and degrees of activation will be mediated by chemokines. The use of small molecules to block chemokines is reviewed by Baggiolini and Moser (1997) J. Exp. Med. 186:1189-1191.

The sequence of chemerin (retinoic acid receptor responder; tazarotene induced) may be found in Genbank, accession number NM_{—}002889. The sequence of CMKLR1 may be found in Genbank, accession number Y14838, and is described by Samson et al. (1998) Eur J. Immunol. 28(5): 1689-700.

### SUMMARY OF THE INVENTION

Methods are provided for modulating leukocyte homing through interactions with one or more of a class of chemoattractant proteins, which chemoattractant proteins are characterized as having a cystatin-like structure. The chemoattractant polypeptides, or mimics thereof, act to concentrate responding leukocytes at a site of interest. Agents that block the activity of the chemoattractant reduce the concentration of leukocytes at a targeted site.

In one embodiment of the invention, the leukocytes are circulating plasmacytoid dendritic cells (pDC), which are shown herein to express the orphan G protein linked receptor CMKLR1, and to migrate in response to its ligand, chemerin. The interaction of chemerin and CMKLR1 coordinate homeostatic trafficking and tissue distribution of pDC.

Localized concentration of chemerin increase the concentration of pDC at the targeted site, and enhance the immune response to an antigen by a mammalian host. The targeted site may be the initial site of immunization where antigen is introduced to the host, or may be secondary sites, such as peripheral lymph nodes and Peyer's patches, where dendritic cell and T cell interactions take place. The methods of the invention are particularly useful in situations where the host response to the antigen is sub-optimal, for example in conditions of chronic infection, a lack of immune response to tumor antigens, and the like. In one aspect of the invention, the antigen is a tumor antigen, and is used to enhance the host immune response to tumor cells present in the body.

In another embodiment, a chemoattractant polypeptide, or agonist of the chemoattractant receptor, is co-administered with an antigen of interest, to enhance the immune response to the antigen. The antigen may be conjugated to the chemoattractant, e.g. as a fusion protein, chemical crosslinking, biotin avidin linkage, and the like. Fusion polypeptides can be delivered as a polypeptide, or can be encoded in an open reading frame that is expressible in the tissue of interest, e.g. as a plasmid, viral vector, and the like.

In another embodiment of the invention, the trafficking of leukocytes is prevented by the administration of blocking agents that interfere with the binding of a cystatin-like chemoattractant to its receptor; or compounds that prevent expression of, or signaling through, a cystatin-like chemoattractant receptor.

In some embodiments of the invention, the cystatin-like chemoattractant is provided in an activated form, which activation may include cleavage of 5 or more amino acids at the C-terminus. In another embodiment of the invention, an agent that activates an endogenous cystatin-like chemoattractant is used to modulate leukocyte trafficking, either in vivo or in an in vitro screening assay. Such activating agents include plasmin, plasminogen activators, and other proteases.

In another embodiment of the invention, in vitro derived immature dendritic cells are administered to a patient in combination with a chemoattractant of the present invention, in order to regulate the immune responsiveness of the dendritic cells.

### BRIEF DESCRIPTION OF THE DRAWINGS

FIG. 1A-B. CMKLR1 is expressed on immature monocyte-derived dendritic cells. A. Monocytes were cultured for 7 days with GM-CSF and IL-4 (100 ng/ml ea.). In vitro cultured DC down-regulate CD14 upon maturation, while maintaining MHC class II expression throughout differentiation. Immature day 7 DC express CMKLR1, while day 0 monocytes and day 9 LPS-matured DC are negative by staining. B. CMKLR1 receptor expression increases overtime as monocytes differentiated into immature DC (no LPS added).

FIG. 2A-F. CMKLR1 is selectively expressed on blood pDC and down-regulated upon activation. A. A small subset of freshly isolated Lin− (negative for CD3, CD14, CD16, CD19, CD20, CD56) PBMC expresses CMKLR1 (circled population is ˜0.5% total PBMC). B. Circulating CD3+ T cells, CD16+ NK cells, and CD19+ B cells are negative for CMKLR1 expression. C. Blood PBMC were stained with Lin FITC, rat α-CMKLR1 (2° α-rat PE), CD123 Cychrome, mouse anti-HLADR (2° α-mouse APC). Lin-HLADR+blood dendritic cells were subdivided by CD123 expression. Lin-HLADR+CD123+ plasmacytoid DC stained for CMKLR1, while Lin-HLADR+CD123− myeloid DC were negative for the receptor, n=13 different blood donors. D. Blood PBMC were stained with Lin FITC, CD11c APC, BDCA2 or BDCA4 (2° α-mouse Cychrome). Lin-CD11c−BDCA2+ and Lin-CD11c−BDCA4+ pDC were CMKLR1+ (horizontal line indicates isotype control staining levels), n=3. E. CMKLR1+Lin− cells, pDC, and mDC (Lin−HLADR+CD123+CD11c− and CD123-CD11c+, respectively), were sorted, harvested by cytospin, and stained by Wright-Giemsa. Cells were examined by light microscope using a 40× objective. F. PBMC were cultured overnight with either no stimulation or CpG oligonucleotides, and the pDC were examined for CMKLR1 expression (in separate experiments, CD83 up-regulation was used to demonstrate pDC activation by CpG), n=3.

FIG. 3A-D. Identification of serum chemerin as chemoattractant ligand for CMKLR1. A. 1.6 L of human serum was pre-filtered and applied to a 50 ml heparin sepharose column, and bound protein was eluted using a NaCl gradient. The 0.7M NaCl fraction was enriched for chemotactic activity as assayed by transwell CMKLR1/L1.2 migration. Total protein was determined by BCA. B. Four mass values from the tryptic digest of the isolated chemotactic protein matched four peptides in public databases corresponding to the polypeptide encoded by tazarotene-induced gene 2 (TIG2, or chemerin) [search parameters included 1 missed trypsin cleavage and cysteines modified by acrylamide adducts]. The asterisk-marked peptides were microsequenced by MS/MS fragmentation, and the results were consistent with the predicted peptide sequence. C. Chemerin/L1.2 transfectants were generated and varying dilutions (1:60, 1:6, 1:3) of conditioned media (CM) were tested for chemotactic activity. CM was generated by culturing L1.2 transfectants in low-serum Optimem, harvesting the exhausted media, filtering and concentrating it. A 1:6 dilution of CM from empty vector transfectants (vector CM) was tested as a negative control. Empty vector L1.2 control transfectants did not respond to chemerin CM (not shown). (−) was media alone and (+) serum was a 1:6 dilution of purified human serum. The mean from duplicate wells of a representative experiment with range is presented, n>3. D. Immature DC show a robust migratory response to chemerin CM. The (+) chemokine pool was 10 nM each CXCL12, CCL19, CCL21 (SLC), and CCL2 (MCP-1), and (−) was no chemokine. Each bar represents the mean (+/−sem) percent input migration from 3 experiments (3 different blood donors) performed with duplicate wells. *p<0.05, **p<0.005 by t-test comparing (−) vs. (+) or vector CM vs. chemerin CM.

FIG. 4A-C. Chemerin RNA expression. A. A human RNA array was probed with chemerin cDNA. Chemerin is widely expressed, with highest levels in the adrenal gland, liver, and pancreas, and strong signals in many tissues. Notable exclusions included components of the CNS, BM (bone marrow), PBL (peripheral blood leukocytes), and thymus. B. RNA spot identifier. C. RT-PCR using intron-spanning primers shows chemerin expression in skin, and confirms high levels of expression in the liver (SI is small intestine). Weak expression in the BM is consistent with the dot blot data. G3PDH demonstrates equivalent RNA template in each sample. “No RT” controls showed no amplicons, indicating that the PCR bands reflect RNA expression.

FIG. 5. Serum but not plasma attracts CMKLR1 transfectants. For the “blood plasma” and “blood serum” samples, normal or anti-coagulated blood was collected from the same donor and incubated at RT. At the indicated time points, plasma and serum were clarified by centrifugation, placed on ice, and tested for attractant activity with CMKLR1 transfectants at 1:17 dilution. In comparing attractant activity, an equivalent amount of anticoagulant was added to each serum sample before testing to control for the anticoagulant present in the plasma samples. For the “cell free serum” sample, normal blood was collected from the same donor, immediately centrifuged, and the fluid phase was collected and incubated at RT. At the indicated time points, heparin was added to arrest coagulation, and the samples were placed on ice and then tested in chemotaxis as above. The mean from duplicate wells of a representative experiment is presented with range, n>5 donors; o/n is overnight.

FIG. 6. Chemerin is a potent chemoattractant for human blood pDC. Transendothelial migration was investigated using transwell inserts coated with HUVEC monolayers. Total PBMC were tested, and the migrated cells were collected and stained for HLADR, CD123, CD11c, and Lin markers. 20 nM CXCL12 was used as a positive control, the CCR7 ligands CCL19 (100 nM) and CCL21 (10 nM) were assayed, and the pro-inflammatory chemokine CXCL9 (100 nM) was also tested. A range of concentrations of recombinant bacterial-expressed chemerin was assayed, n=3 donors, mean±S.E. % migration of plasmacytoid or myeloid DC is displayed, *p<0.05, **p<0.005 in pairwise comparisons with background migration (−).

### DESCRIPTION OF THE SPECIFIC EMBODIMENTS

In the subject methods, compounds that modulate the triggering activity of cystatin-like chemoattractants are administered systemically or locally to alter the trafficking behavior of leukocytes. Trafficking, or homing, is used herein to refer to the biological activities and pathways that control the localization of leukocytes in a mammalian host. Such trafficking may be associated with disease, e.g. inflammation, allergic reactions, etc., or may be part of normal biological homeostasis.

Local administration that provides for a prolonged localized concentration, which may utilize sustained release implants or other topical formulation, is of particular interest. In one embodiment of the invention the modulating compound is cystatin-like chemoattractant or an agonist thereof, which acts to increase the local concentration of responsive leukocytes. In an alternative embodiment, the modulating compound blocks activity of a cystatin-like chemoattractant, and decreases leukocyte trafficking. In vivo uses of the method are of interest for therapeutic and investigational purposes. In vitro uses are of interest for drug screening, determination of physiological pathways, and the like.

T-regulatory (Tr) cells suppress the immune response and have been demonstrated to prevent autoimmune pathologies, allograft transplant rejection, and graft-versus-host disease (GVHD). The generation of Tr cells is antigen dependent and can be induced by immature DC (in vitro monocyte-derived) as well as by pDC treated with CD40L and IL3. Direct cell-cell contact, as well as secretion of IL10 by Tr cells, has been shown to dampen the immune response. These tolerogenice DCs may be pulsed in vitro with self-antigens (in the case of autoimmunity) or alloantigens (in the case of transplantation) and delivered to patients, where they may be able to induce Tr cells capable of downregulating self- or allo-reactivity.

Such cells are provided with the appropriate microenvironmental milieu of cytokines, e.g. in the presence of, or in conjunction with cytokines or other agents that maintain the ability of pDC's to induce Tr's, and concentrated by the use of chemerin at a site of interest. In one embodiment of the invention, chemerin is used to localize dendritic cells, particularly immature dendritic cells or pDC, in order to alleviate the immune responsiveness at a site, e.g. at a site of transplantation, at a site of active autoimmune disease, and the like, in order to dampen an immune response.

Chemoattractants, as used herein, include the cystatin-like chemoattractant polypeptides; and functional equivalents thereof, for example compounds that bind to the chemoattractant receptor and activate signaling pathways activated by the native polypeptide. Such agents typically interact with the extracellular binding domain or transmembrane domain of a receptor protein, and may activate the receptor through the ligand binding site, block the ligand binding site, conformationally alter the receptor, etc. Antagonists, or inhibitors, are molecules that specifically act to block the chemoattractant activity. Such inhibitors may act to interfere with the interaction between a cystatin-like chemoattractant and receptor, or may directly interfere with the receptor.

Cystatins have 2 disulfide bonds, a signature “cystatin-fold”, which is defined as a core structure having an alpha helix packed against a coiled beta sheet made up of 4 anti-parallel beta strands. Cystatins are secreted and found in most body fluids. In contrast, stefins have no disulfide bonds, are not secreted (cytosolic), and adopt a cystatin-like fold. Kininogens are predicted to have three cystatin-like domains, and are found in blood plasma.

The family of cystatin-like chemoattractants include the polypeptides: Cst1 (Genbank accession number NM_{—}001898); Cst2 (Genbank accession number NM_{—}001322); Cst3 (Genbank accession number NM_{—}000099); Cst4 (Genbank accession number NM_{—}001899); Cst5 (Genbank accession number NM_{—}001900); Cst6 (Genbank accession number NM_{—}001323); Cst7 (Genbank accession number NM_{—}003650); Cst8 (Genbank accession number NM_{—}005492); Cst9-like (Genbank accession number NM_{—}080610); Cst11 variant 1 (Genbank accession number NM_{—}130794); Cst11 variant 2 (Genbank accession number NM_{—}08030); CstA (or stefin A) (Genbank accession number BC010379); CstB (or stefin B) (Genbank accession number BT007040); Kininogen (Genbank accession number NM_{—}000893); Fetuin B (Genbank accession number NM_{—}014375); Cathelicidin LL37 (Genbank accession number NM_{—}004345); Cathepsin F (Genbank accession number AF088886); Invariant chain (Ii) (Genbank accession number NP_{—}004346); gamma-glutamyltransferase-like activity 3 (GGTLA3) (Genbank accession number XM_{—}066189); and histidine-rich glycoprotein (Genbank accession number M13149). In some embodiments of the invention, the chemoattractant is other than Cathelicidin LL37 or chemerin. In other embodiments, the chemoattractant is chemerin (chemerin) Genbank accession number NM_{—}002889, which encodes the polypeptide: MRRLLIPLALWLGAVGVGVAELTEAQRRGLQVALEEFHKHPPVQWAFQETSVESAVDTPFPAGIFVR LEFKLQQTSCRKRDWKKPECKVRPNGRKRKCLACIKLGSEDKVLGRLVHCPIETQVLREAEEHQETQ CLRVQRAGEDPHSFYFPGQFAFSKALPRS.

Chemoattractant receptors of interest include CMKLR1, Genbank accession number Y14838; GPR1, Genbank accession number NM_{—}005279; GPRW, Genbank accession number AF045764; GPR44, Genbank accession number AF118265; MAS1, Genbank accession number Ml 3150; and MRG1, Genbank accession number S78653. Other chemoattractant receptors of interest include formyl-peptide receptor (FPR): NM_{—}002029; formyl-peptide receptor-like 1 (FPRL1): NM_{—}001462; formyl-peptide receptor-like 2 (FPRL2): NM_{—}002030; CRTH2 (which is a truncated version of GPR44): AB008535; complement 5a (C5a) receptor: NP_{—}001727; and complement 3a (C3a) receptor: NM_{—}004054.

### Chemoattractant Polypeptides

Cystatin-like chemoattractant polypeptides are of interest for concentrating responding leukocytes, screening methods, as reagents to raise antibodies, as therapeutics, and the like. Such polypeptides can be produced through isolation from natural sources, recombinant methods and chemical synthesis. In addition, functionally equivalent polypeptides may find use, where the equivalent polypeptide may contain deletions, additions or substitutions of amino acid residues that result in a silent change, thus producing a functionally equivalent differentially expressed on pathway gene product. Amino acid substitutions may be made on the basis of similarity in polarity, charge, solubility, hydrophobicity, hydrophilicity, and/or the amphipathic nature of the residues involved. “Functionally equivalent”, as used herein, refers to a protein capable of exhibiting a substantially similar in vivo activity.

The sequence of the polypeptides may be altered in various ways known in the art to generate targeted changes in sequence. A variant polypeptide will usually be substantially similar to the sequences provided herein, i.e. will differ by at least one amino acid, and may differ by at least two but usually not more than about four amino acids. The sequence changes may be substitutions, insertions or deletions. Scanning mutations that systematically introduce alanine, or other residues, may be used to determine key amino acids. Amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine). The polypeptides may be also be altered, e.g. by mutagenesis, in order to alter the biological activity. Such alterations may be tested by screening assays, as described below, in order to determine if a particular mutation results in increased agonist activity, decreased agonist activity, increased antagonist activity, or decreased antagonist activity.

Modifications of interest also include activation of the chemoattractant, e.g. by enzymatic cleavage at the C-terminal, which cleavage may include processing by serine proteases, e.g. enzymes in the coagulation or fibrinolytic cascades. Such enzymes may also be used to modulate leukocyte trafficking through the activation of endogenous chemoattractants. For example, plasmin treatment of normal blood or serum significantly enhances the endogenous chemoattractant activity. Administration of such enzymes, e.g. in a targeted method of drug delivery, may be used to enhance leukocyte trafficking.

Modifications of interest that do not alter primary sequence include chemical derivatization of polypeptides, e.g., acetylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids.

As an option to recombinant methods, polypeptides and oligopeptides can be chemically synthesized. Such methods typically include solid-state approaches, but can also utilize solution based chemistries and combinations or combinations of solid-state and solution approaches. Examples of solid-state methodologies for synthesizing proteins are described by Merrifield (1964) J. Am. Chem. Soc. 85:2149; and Houghton (1985) Proc. Natl. Acad. Sci., 82:5132. Fragments of cystatin-like chemoattractant protein can be synthesized and then joined together. Methods for conducting such reactions are described by Grant (1992) Synthetic Peptides: A User Guide, W.H. Freeman and Co., N.Y.; and in “Principles of Peptide Synthesis,” (Bodansky and Trost, ed.), Springer-Verlag, Inc. N.Y., (1993).

If desired, various groups may be introduced into the peptide during synthesis or during synthesis, which allow for linking to other molecules or to a surface. Thus, cysteines can be used to make thioethers, histidines for linking to a metal ion complex, carboxyl groups for forming amides or esters, amino groups for forming amides, and the like.

The polypeptides may be produced by recombinant DNA technology using techniques well known in the art. Methods that are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized.

Typically, the coding sequence is placed under the control of a promoter that is functional in the desired host cell to produce relatively large quantities of the gene product. An extremely wide variety of promoters are well-known, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Expression can be achieved in prokaryotic and eukaryotic cells utilizing promoters and other regulatory agents appropriate for the particular host cell. Exemplary host cells include, but are not limited to, E. coli, other bacterial hosts, yeast, and various higher eukaryotic cells such as the COS, CHO and HeLa cells lines and myeloma cell lines.

In mammalian host cells, a number of viral-based expression systems may be used, including retrovirus, lentivirus, adenovirus, adeno-associated virus, and the like. In cases where an adenovirus is used as an expression vector, the coding sequence of interest can be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing differentially expressed or pathway gene protein in infected hosts.

Specific initiation signals may also be required for efficient translation of the genes. These signals include the ATG initiation codon and adjacent sequences. In cases where a complete gene, including its own initiation codon and adjacent sequences, is inserted into the appropriate expression vector, no additional translational control signals may be needed. However, in cases where only a portion of the gene coding sequence is inserted, exogenous translational control signals must be provided. These exogenous translational control signals and initiation codons can be of a variety of origins, both natural and synthetic. The efficiency of expression may be enhanced by the inclusion of appropriate transcription enhancer elements, transcription terminators, etc.

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38, etc.

For long-term, high-yield production of recombinant proteins, stable expression is preferred. For example, cell lines that stably express the differentially expressed or pathway gene protein may be engineered. Rather than using expression vectors that contain viral origins of replication, host cells can be transformed with DNA controlled by appropriate expression control elements, and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines that express the target protein. Such engineered cell lines may be particularly useful in screening and evaluation of compounds that affect the endogenous activity of the cystatin-like chemoattractant protein. A number of selection systems may be used, including but not limited to the herpes simplex virus thymidine kinase, hypoxanthine-guanine phosphoribosyltransferase, and adenine phosphoribosyltransferase genes. Antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin.

The polypeptide may be labeled, either directly or indirectly. Any of a variety of suitable labeling systems may be used, including but not limited to, radioisotopes such as ¹²⁵I; enzyme labeling systems that generate a detectable colorimetric signal or light when exposed to substrate; and fluorescent labels. Indirect labeling involves the use of a protein, such as a labeled antibody, that specifically binds to the polypeptide of interest. Such antibodies include but are not limited to polyclonal, monoclonal, chimeric, single chain, Fab fragments and fragments produced by a Fab expression library.

Once expressed, the recombinant polypeptides can be purified according to standard procedures of the art, including ammonium sulfate precipitation, affinity columns, ion exchange and/or size exclusivity chromatography, gel electrophoresis and the like (see, generally, R. Scopes, Protein Purification, Springer—Verlag, N.Y. (1982), Deutscher, Methods in Enzymology Vol. 182: Guide to Protein Purification., Academic Press, Inc. N.Y. (1990)).

For various purposes, for example as an immunogen, the entire cystatin-like chemoattractant polypeptide or a fragment derived therefrom may be used. Preferably, one or more 8-30 amino acid peptide portions, e.g. of an extracellular domain may be utilized. Custom-synthesized peptides in this range are available from a multitude of vendors, and can be order conjugated to KLH or BSA. Alternatively, peptides in excess of 30 amino acids may be synthesized by solid-phase methods, or may be recombinantly produced in a suitable recombinant protein production system. In order to ensure proper protein glycosylation and processing, an animal cell system (e.g., Sf9 or other insect cells, CHO or other mammalian cells) is preferred.

### Chemoattractant Agonists and Antagonists

Candidate cystatin-like chemoattractant antagonists or agonists may be identified by detecting the ability of an agent to affect the interaction of a cystatin-like chemoattractant with it's cognate receptor, as described below. Agonists typically bind to, and activate the receptor of interest, while antagonists block the binding between the chemoattractant and its receptor.

Agents of interest include chemoattractants, mimics, and inhibitors, and may be peptides, small organic molecules, peptidomimetics, soluble T cell receptors, antibodies, or the like. Antibodies are an exemplary agent for inhibiting chemotaxis, and for acting as chemoattractant mimic, mimicking the chemoattractant activity of the cystatin-like polypeptide. Antibodies may be polyclonal or monoclonal; intact or truncated, e.g. F(ab′)₂, Fab, Fv; xenogeneic, allogeneic, syngeneic, or modified forms thereof, e.g. humanized, chimeric, etc.

In many cases, the agent will be an oligopeptide, e.g. antibody or fragment thereof, etc., but other molecules that provide relatively high specificity and affinity may also be employed. Combinatorial libraries provide compounds other than oligopeptides that have the necessary binding characteristics. Generally, the affinity will be at least about 10⁻⁶, more usually about 10⁻⁸ M, i.e. binding affinities normally observed with specific monoclonal antibodies.

Candidate agents are screened for their ability to meet this criteria. Assays to determine affinity and specificity of binding are known in the art, including competitive and non-competitive assays. Assays of interest include ELISA, RIA, flow cytometry, etc. Binding assays may use purified or semi-purified protein, or alternatively may use native leukocytes that express a receptor of interest, or other cells, e.g. cells transfected with an expression construct for a cystatin-like chemoattractant receptor; membranes from these cells; etc. As an example of a binding assay, cystatin-like chemoattractant receptor that is inserted in a membrane, e.g. whole cells, or membranes coating a substrate, e.g. microtiter plate, magnetic beads, etc. The candidate agent and soluble, labeled chemoattractant are added to the cells, and the unbound components are then washed off. The ability of the modulating agent to compete with a chemoattractant for receptor binding is determined by quantitation of bound, labeled chemoattractant polypeptide. Confirmation that the blocking agent does not cross-react with other chemokine receptors may be performed with a similar assay.

Cystatin-like chemoattractant protein sequences are used in screening of candidate compounds, including antibodies and small organic molecules, for the ability to bind to and/or inhibit cystatin-like chemoattractant activity. Agents that inhibit cystatin-like chemoattractants are of interest as therapeutic agents decreasing leukocyte trafficking, while mimics are of interest for enhancing an immune response. Such compound screening may be performed using an in vitro model, a genetically altered cell or animal, or purified protein corresponding to cystatin-like chemoattractant polypeptides or a fragment thereof. One can identify ligands or substrates that bind to, modulate or mimic the action of the encoded polypeptide.

Polypeptides useful in screening include those encoded by the cystatin-like chemoattractant gene, as well as nucleic acids that, by virtue of the degeneracy of the genetic code, are not identical in sequence to the disclosed nucleic acids, and variants thereof.

Transgenic animals or cells derived therefrom are also used in compound screening. Transgenic animals may be made through homologous recombination, where the normal locus corresponding to cystatin-like chemoattractant is altered. Alternatively, a nucleic acid construct is randomly integrated into the genome. Vectors for stable integration include plasmids, retroviruses and other animal viruses, YACs, and the like. A series of small deletions and/or substitutions may be made in the coding sequence to determine the role of different exons in receptor binding, signal transduction, etc. Specific constructs of interest include antisense sequences that block expression of the targeted gene and expression of dominant negative mutations. A detectable marker, such as lac Z may be introduced into the locus of interest, where up-regulation of expression will result in an easily detected change in phenotype. One may also provide for expression of the target gene or variants thereof in cells or tissues where it is not normally expressed or at abnormal times of development, for example by overexpressing in neural cells. By providing expression of the target protein in cells in which it is not normally produced, one can induce changes in cell behavior.

Compound screening identifies agents that modulate chemoattractant function. Of particular interest are screening assays for agents that have a low toxicity for normal human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains and sites.

The term “agent” as used herein describes any molecule, e.g. protein or pharmaceutical, with the capability of altering or mimicking the physiological function of cystatin-like chemoattractant protein. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of phosphatase inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

A “combinatorial library” is a collection of compounds in which the compounds comprising the collection are composed of one or more types of subunits. Methods of making combinatorial libraries are known in the art, and include the following: U.S. Pat. Nos. 5,958,792; 5,807,683; 6,004,617; 6,077,954; which are incorporated by reference herein. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules that are different from each other. Each of the different molecules is present in a detectable amount. The actual amounts of each different molecule needed so that its presence can be determined can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, hydrocarbon substituents, e.g. aliphatic, alicyclic substituents, aromatic, aliphatic and alicyclic-substituted aromatic nuclei, and the like, as well as cyclic substituents; substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like); and hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to chemoattractants; compounds so identified are possible inhibitors. Compounds capable of binding to chemoattractant receptors may be inhibitors if they do not activate the receptor, and may be mimics if they do activate the receptor. The binding assays usually involve contacting cystatin-like chemoattractant or receptor with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89.

Certain screening methods involve screening for a compound that modulates the expression of a cystatin-like chemoattractant. Such methods generally involve conducting cell-based assays in which test compounds are contacted with one or more cells expressing cystatin-like chemoattractant and then detecting and an increase in expression. The level of expression or activity can be compared to a baseline value. The baseline value can be a value for a control sample or a statistical value that is representative of expression levels for a control population. Expression levels can also be determined for cells that do not express the chemoattractant, as a negative control. Such cells generally are otherwise substantially genetically the same as the test cells. Various controls can be conducted to ensure that an observed activity is authentic including running parallel reactions with cells that lack the reporter construct or by not contacting a cell harboring the reporter construct with test compound. Compounds can also be further validated as described below.

Compounds that are initially identified by any of the foregoing screening methods can be further tested to validate the apparent activity. The basic format of such methods involves administering a lead compound identified during an initial screen to an animal that serves as a model for humans. The animal models utilized in validation studies generally are mammals. Specific examples of suitable animals include, but are not limited to, primates, mice, and rats.

Active test agents identified by the screening methods described herein that inhibit or mimic chemotaxis can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

A functional assay that detects leukocyte chemotaxis may be used for confirmation. For example, a population of dendritic cells may be stimulated with chemerin, in the presence or absence of the candidate modulating agent. An agent that blocks chemotaxis will cause a decrease in the dendritic cell locomotion, as measured by the assays described in the examples provided herein, etc. An agent that is a chemoattractant mimic will increase concentration of dendritic cells at a target site of higher concentration; and an inhibitor will block such an increase in concentration.

### Antibodies

In some embodiments, the cystatin-like chemoattractant agonist or antagonist is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The term includes monoclonal antibodies, multispecific antibodies (antibodies that include more than one domain specificity), human antibody, humanized antibody, and antibody fragments with the desired biological activity.

The specific or selective fit of a given structure and its specific epitope is sometimes referred to as a “lock and key” fit. The archetypal antibody molecule is the immunoglobulin, and all types of immunoglobulins, IgG, e.g. IgG1, IgG2a, IgG2b, IgG3, IgG4, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be polyclonal antibodies, although monoclonal antibodies are preferred because they may be reproduced by cell culture or recombinantly, and can be modified to reduce their antigenicity. Such antibodies are well known in the art and commercially available, for example from Research Diagnostics, Becton Dickinson, etc.

Polyclonal antibodies can be raised by a standard protocol by injecting a production animal with an antigenic composition, formulated as described above. See, e.g., Harlow and Lane, Antibodies: A Laboratory Manual, Cold Spring Harbor Laboratory, 1988. In one such technique, a cystatin-like chemoattractant, or receptor, or an antigenic portion of the polypeptide thereof, is initially injected into any of a wide variety of mammals (e.g., mice, rats, rabbits, sheep or goats). When utilizing an entire protein, or a larger section of the protein, antibodies may be raised by immunizing the production animal with the protein and a suitable adjuvant (e.g., Fruend's, Fruend's complete, oil-in-water emulsions, etc.) When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions, a superior immune response may be elicited if the polypeptide is joined to a carrier protein, such as ovalbumin, BSA or KLH. The peptide-conjugate is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. The immortal cell line utilized is preferably selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

Preferably, the immortal fusion partners utilized are derived from a line that does not secrete immunoglobulin. The resulting fused cells, or hybridomas, are cultured under conditions that allow for the survival of fused, but not unfused, cells and the resulting colonies screened for the production of the desired monoclonal antibodies. Colonies producing such antibodies are cloned, expanded, and grown so as to produce large quantities of antibody, see Kohler and Milstein, 1975 Nature 256:495 (the disclosures of which are hereby incorporated by reference).

Large quantities of monoclonal antibodies from the secreting hybridomas may then be produced by injecting the clones into the peritoneal cavity of mice and harvesting the ascites fluid therefrom. The mice, preferably primed with pristane, or some other tumor-promoter, and immunosuppressed chemically or by irradiation, may be any of various suitable strains known to those in the art. The ascites fluid is harvested from the mice and the monoclonal antibody purified therefrom, for example, by CM Sepharose column or other chromatographic means. Alternatively, the hybridomas may be cultured in vitro or as suspension cultures. Batch, continuous culture, or other suitable culture processes may be utilized. Monoclonal antibodies are then recovered from the culture medium or supernatant.

In addition, the antibodies or antigen binding fragments may be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones, which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell). When antibody gene synthesis is induced in the transfected host, the heavy and light chain proteins self-assemble to produce active antibodies that can be detected by screening with the antigen or immunogen.

Antibodies with a reduced propensity to induce a violent or detrimental immune response in humans (such as anaphylactic shock), and which also exhibit a reduced propensity for priming an immune response which would prevent repeated dosage with the antibody therapeutic or imaging agent are preferred for use in the invention. Thus, humanized, single chain, chimeric, or human antibodies, which produce less of an immune response when administered to humans, are preferred for use in the present invention. Also included in the invention are multi-domain antibodies.

A chimeric antibody is a molecule in which different portions are derived from different animal species, for example those having a variable region derived from a murine mAb and a human immunoglobulin constant region. Techniques for the development of chimeric antibodies are described in the literature. See, for example, Morrison et al. (1984) Proc. Natl. Acad. Sci. 81:6851-6855; Neuberger et al. (1984) Nature 312:604-608; Takeda et al. (1985) Nature 314:452-454. Single chain antibodies are formed by linking the heavy and light chain fragments of the Fv region via an amino acid bridge, resulting in a single chain polypeptide. See, for example, Huston et al., Science 242:423-426; Proc. Natl. Acad. Sci. 85:5879-5883; and Ward et al. Nature 341:544-546.

Antibody fragments that recognize specific epitopes may be generated by techniques well known in the field. These fragments include, without limitation, F(ab′)₂ fragments, which can be produced by pepsin digestion of the antibody molecule, and Fab fragments, which can be generated by reducing the disulfide bridges of the F(ab′)₂ fragments.

In one embodiment of the invention, a human or humanized antibody is provided, which specifically binds to the extracellular region of cystatin-like chemoattractant receptor with high affinity. In another embodiment, a human or humanized antibody is provided, which specifically binds to the cystatin-like chemoattractant.

Alternatively, polyclonal or monoclonal antibodies may be produced from animals that have been genetically altered to produce human immunoglobulins. Techniques for generating such animals, and deriving antibodies therefrom, are described in U.S. Pat. No. 6,162,963 and 6,150,584, incorporated fully herein by reference.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

Candidate anti-cystatin-like chemoattractant, or receptor, antibodies can be tested for by any suitable standard means, e.g. ELISA assays, etc. As a first screen, the antibodies may be tested for binding against the immunogen, or against the entire polypeptide. As a second screen, anti-cystatin-like chemoattractant candidates may be tested for binding to an tissue expressing a receptor. For these screens, the anti- cystatin-like chemoattractant candidate antibody may be labeled for detection. After selective binding is established, the candidate antibody, or an antibody conjugate may be tested for appropriate activity (i.e., the ability to increase local concentration of a leukocyte of interest, or to block chemotaxcis) in an in vivo model. In a preferred embodiment, anti- cystatin-like chemoattractant protein compounds may be screened using a variety of methods in vitro and in vivo. These methods include, but are not limited to, methods that measure binding affinity to a target, biodistribution of the compound within an animal or cell, etc. These and other screening methods known in the art provide information on the ability of a compound to bind to, modulate, or otherwise interact with the specified target and are a measure of the compound's efficacy.

### Leukocytes

Cells of interest for modulation by the methods of the invention include dendritic cells, particularly plasmacytoid dendritic cells. These cells are key producers of type I interferons, cytokines that can directly block viral replication and stimulate the adaptive immune response. Following activation by virus, unmethylated bacterial DNA (mimicked by oligonucleotide CpG) or CD40L+IL3, pDCs mature into potent antigen presenting cells (APCs), as defined by their ability to stimulate naïve allogeneic CD4⁺ T cell proliferation. pDCs have been characterized primarily in peripheral blood and are identified as Lin⁻HLADR⁺CD123⁺CD11c⁻; they are also positive for 2 recently identified pDC-selective antigens, BDCA2 (a C-type lectin) and BDCA4 (neuropilin-1).

Dendritic cells are a class of “professional” antigen presenting cells, and have a high capacity for sensitizing MHC-restricted T cells. They are typically characterized by expression of MHC class II (HLADR+), and lack of expression of “lineage” markers (Lin negative defined as non-lymphocyte, non-monocyte, i.e. CD3-CD14-CD16-CD19-CD20-CD56-). Mature dendritic cells typically express higher levels of costimulatory molecules such as CD40, CD80, and CD86, as well as MHC class II, than immature DC. Precursor DC can have the phenotype CD11c⁻, CD123^{low}; and those that are CD11c^{-high} (pDC precursors). Treatment with GM-CSF in vivo preferentially expands myeloid-type CD11c^{high} DC, while Flt-3 ligand has been shown to expand both myeloid-type CD11c⁺ CD123^{low} DC, and plasmacytoid-type CD11c⁻ CD123^{high} DC precursors.

Other leukocyte cells of interest for the present methods include polymorphonuclear cells, e.g. basophils, eosinophils, and neutrophils. One aspect of the invention is the effect of modulating polymorphonuclear leukocytes (PMN) trafficking, e.g. in locomotion in extravascular tissue. PMNs include neutrophils, which are primarily found in storage in the bone marrow. The major inflammatory functions of neutrophils include phagocytosis and secretion of pro-inflammatory substances. As a general rule, neutrophils are the predominant cell type in acute inflammation. Pro-inflammatory substances released by neutrophils include lysosomal enzymes, products of oxygen metabolism, and products of arachidonic acid metabolism.

Eosinophils are prominent at sites of allergic reactions, and with parasitic infections. Eosinophil secretory products inactivate many of the chemical mediators of inflammation. This phenomenon is most obvious with mast cell-derived mediators. Mast cells produce a chemotactic factor for eosinophils. Secretory products of eosinophils can kill parasitic larvae by disrupting their cuticles, and parasite-induced IgE-containing immune complexes are chemotactic for eosinophils.

Basophils are the circulating counterpart of mast cells, and are often associated with allergic reactions and parasitic infections. Their major inflammatory function is release of basophil granule contents that incite vascular changes at sites of acute inflammation. Increased numbers of basophils are located in skin affected with ectoparasites.

Mononuclear cells are also of interest, including mononuclear phagocytes and mast cells. Another aspect of the invention is the modulation of monocyte trafficking. Monocytes are of interest as immune effector cells, and as antigen presenting cells. The administration of agents that block chemoattractants decreases the trafficking of monocytes to sites of inflammation, and the administration of activating agents may enhance monocyte trafficking.

The mononuclear phagocyte system is comprised of both circulating and fixed populations of cells. The circulating component is the monocyte. Upon migration into tissues these are referred to as histiocytes or tissue macrophages. The major fixed macrophages include: Sinusoidal lining cells of the spleen, lymph nodes, liver, and bone marrow; connective tissue histiocytes; mobile macrophages on serosal surfaces; alveolar macrophages within the lung; microglia in the nervous system; and mesangial macrophages within renal glomeruli. Macrophages produce a variety of substances that are involved in inflammation. Mast cells are important mediators of certain allergic reactions. Mast cell membranes have abundant IgE receptor sites, anywhere from 30,000 to 500,000 per cell. If a particular antigen incites an IgE response, the resulting IgE is bound to the IgE receptors on mast cell surfaces via the Fc portion of the immunoglobulin molecule. Interaction of an antigen with surface-bound IgE results in cross-linking of the IgE molecules, mast cell activation, and ultimately mast cell degranulation.

Lymphocytes are another class on mononuclear cell of interest for the methods of the invention. Lymphocytes may be broadly divided into B cells, T cells and natural killer cells. T cells and B cells are able to give rise to memory cells, as well as effector cells.

The B cell lineage includes pre-B cells, B cells and plasma cells, which are the terminal immunoglobulin (Ig) producing cell. Initially, B cells express immunoglobulin of the IgM class, but during differentiation and interaction with T cells, there is usually a class switch to Ig of other classes, including the human classes: IgM, IgG1, IgG2, IgG3, IgG4, IgA1, IgA2, and IgE. IgA is found in secretions, e.g. saliva, milk, mucus, etc., and IgA expressing B cells are expected to concentrate in secretory organs. Other classes of B cells or plasma cells, including memory cells, may be expected to respond to chemoattractants appropriate for the class of immunoglobulin and antigenic specificity.

Circulating T cells are small, round-shaped cells with very little cytoplasm and a number of protrusions (microvilli) on the plasma membrane. During extravasation, T cells undergo major morphological changes as they adhere, spread and eventually transmigrate through the endothelium. The recirculation pattern of T cells is highly regulated by the modulated expression and function of specific receptor-ligand pairs on the cell surface of T and endothelial cells, respectively.

Naive T cells are primed by specialized antigen presenting cells in secondary lymphoid tissues. Upon antigen recognition they undergo clonal amplification and progressively acquire differentiated functions. Cytolytic T cells are CD8+, and can secrete a number of lytic proteins. CD4⁺ T cells mature into two major subsets of effectors, based on the cytokines they produce. Th1 and Th2 cells enhance cellular and humoral adaptive responses to antigen. A third subset comprises T regulatory cells (Tr), which negatively control the above responses due to the production of selected cytokines.

Maturation of T cells includes the acquisition of a memory phenotype by a subpopulation of clonally expanded T cells that progressively exit the cell cycle and revert to a quiescent state. Memory may be long-lasting, and is both antigenic and topographic, the latter being provided by the expression of defined arrays of chemotactic and homing receptors. These dictate the recirculation pattern of memory versus naive T cells. To ensure maximal efficiency and sensitivity in antigen recognition and elimination, naive cells preferentially recirculate through secondary lymphoid organs, while memory and effector cells patrol peripheral tissues and re-enter the blood via the afferent lymphatics.

### Methods of Use

The specific immune response for an antigen of interest is enhanced by increasing the site specific concentration of dendritic cells. A cystatin-like chemoattractant agonist that attracts dendritic cells is introduced at the target site, where the target site may be the site of immunization, or a secondary lymphoid organ, e.g. Peyer's patches, lymph nodes, etc. The cystatin-like chemoattractant agonist may be selected to enhance specific subsets of dendritic cells, e.g. activated dendritic cells, precursor dendritic cells, etc. The methods may further be practiced in conjunction with the expansion of functional dendritic cells in vivo, for example through administration of Flt3-L, GM-CSF, and the like.

In another embodiment of the invention, methods are provided to specifically modulate the homing of leukocytes other than dendritic cells, which are responsive to a cystatin-like chemoattractant. Leukocytes expressing a receptor for a cystatin-like chemoattractant concentrate at a target site where the chemoattractant is present. This arrest acts to localize the cells at the target site. Compounds that modulate the chemotaxis of leukocytes are administered systemically or locally to alter the homing behavior of the leukocytes. In one embodiment of the invention the agent is a chemoattractant or chemoattractant mimic, which acts to enhance the chemotaxis effect. In an alternative embodiment, the agent blocks chemotaxis activity.

For methods of enhancing an immune response, an antigen of interest may be delivered to peripheral tissues, e.g. skin, muscle, etc. or other localized sites, e.g. lymph nodes, Peyer's patches, etc., and may be given as a combined formulation, or as separate formulations. The antigen may be further provided in a booster dose, in combination with other adjuvants as known in the art, etc. The methods of the invention are particularly useful in situations where the host response to the antigen is sub-optimal, for example in conditions of chronic infection, a lack of immune response to tumor antigens, and the like.

Mammalian species that may require enhancement of T cell mediated immune responses include canines; felines; equines; bovines; ovines; etc. and primates, particularly humans. Animal models, particularly small mammals, e.g. murine, lagomorpha, etc. may be used for experimental investigations. Animal models of interest include those involved with the immune responses to infection and tumors.

The cystatin-like chemoattractant agonist may be delivered as a bolus, or may provide for a localized concentration by use of a sustained release formulation. For example, it may be desirable to increase the number of dendritic cells at a target site prior to antigen administration. Alternatively, the antigen and localization factor may be co-administered. Preliminary doses can be determined according to animal tests, and the scaling of dosages for human administration can be performed according to art-accepted practices.

A variety of sustained release formulations are known and used in the art. For example, biodegradable or bioerodible implants may be used. The implants may be particles, sheets, patches, plaques, fibers, microcapsules and the like and may be of any size or shape compatible with the selected site of insertion. Characteristics of the polymers will include biodegradability at the site of implantation, compatibility with the agent of interest, ease of encapsulation, the half-life in the physiological environment, water solubility, and the like.

Another approach involves the use of an implantable drug delivery device. Examples of such implantable drug delivery devices include implantable diffusion systems (see, e.g., subdermal implants (such as NORPLANTJ) and other such systems, see, e.g., U.S. Pat. Nos. 5,756,115; 5,429,634; 5,843,069). These implants generally operate by simple diffusion, e.g., the active agent diffuses through a polymeric material at a rate that is controlled by the characteristics of the active agent formulation and the polymeric material. Alternatively, the implant may be based upon an osmotically-driven device to accomplish controlled drug delivery (see, e.g., U.S. Pat. Nos. 3,987,790, 4,865,845; 5,057,318; 5,059,423; 5,112,614; 5,137,727; 5,234,692; 5,234,693; and 5,728,396). These osmotic pumps generally operate by imbibing fluid from the outside environment and releasing corresponding amounts of the therapeutic agent.

In combination with the local recruitment of dendritic cells, the overall number of functionally mature dendritic cells in the host may be expanded through the prior administration of a suitable growth factor, which growth factor may be one or more of Flt3-L; GM-CSF; G-CSF; GM-CSF+IL-4; GM-CSF+IL-3; etc.

For example, Flt3-L has been found to stimulate the generation of large numbers of functionally mature dendritic cells, both in vivo and in vitro (U.S. Ser. No. 08/539,142, filed Oct. 4, 1995). Flt3-L refers to a genus of polypeptides that are described in EP 0627487 A2 and in WO 94/28391, both incorporated herein by reference. A human Flt3-L cDNA was deposited with the American Type Culture Collection, Rockville, Md., USA (ATCC) on Aug. 6, 1993 and assigned accession number ATCC 69382. Other useful cytokines include granulocyte-macrophage colony stimulating factor (GM-CSF; described in U.S. Pat. Nos. 5,108,910, and 5,229,496 each of which is incorporated herein by reference). Commercially available GM-CSF (sargramostim, Leukine®) is obtainable from Immunex Corp., Seattle, Wash.) Moreover, GM-CSF/IL-3 fusion proteins (i.e., a C-terminal to N-terminal fusion of GM-CSF and IL-3) may be used. Such fusion proteins are well known in the art and are described in U.S. Pat. Nos. 5,199,942; 5,108,910 and 5,073,627, each of which is incorporated herein by reference.

Various routes and regimens for delivery may be used, as known and practiced in the art. For example, where the agent is Flt3-L, the Flt3-L may be administered daily, where the dose is from about 1 to 100 mg/kg body weight, more usually from about 10 to about 50 mg/kg body weight. Administration may be at a localized site, e.g. sub-cutaneous, or systemic, e.g. intraperitoneal, intravenous, etc.

After the host has responded to the cystatin-like chemoattractant agonist, usually from about 3 days to 2 weeks, there is an increased number of DC precursors at the site of interest, e.g. skin, muscle, lungs, etc. These cells may not yet be immunologically mature, but can respond to DC activating agents, which agents include a variety of immunostimulatory compounds. Of particular interest for this purpose are immunostimulatory polynucleotide sequences. The DC activating agent is preferably delivered directly to the peripheral tissues.

The presence of DC precursors in the periphery indicates that that the most effective route for delivering the activating agent may be through a local delivery, particularly dermal, sub-cutaneous and intramuscular administration (see U.S. Pat. No. 5,830,877, Carson et al., issued Nov. 3, 1998). Generally the antigen and the DC activating agent will be delivered to the same site, and may be co-formulated, e.g. mixed together, coadministered, conjugated together, etc.; or formulated separately, depending on the requirements of the specific agents.

A number of DC activating agents are known in the art, including LPS and endotoxins in small doses, alpha interferons, interleukin-1 (see Boraschi et al. (1999) Methods 19(1):108-13), modified tumor necrosis factor, CD40 ligand, poly IC, etc. Of particular interest is the use of immunostimulatory polynucleotide sequences (ISS), which have been shown to be highly effective in the activation of DC, and other antigen presenting cells. The use of these sequences is known in the art, for examples see Bauer et al. (1999) Immunology 97(4):699-705; Klinman et al (1999) Vaccine 17(1):19-25; Hasan et al. (1999) J Immunol Methods 229(1-2):1-22; and others.

Concurrent with the administration of a DC activating agent, or following pDC concentration at a site of interest, antigen is provided in one or more doses. Antigens of interest include polypeptides and other immunogenic biomolecules, which may be isolated or derived from natural sources, produced by recombinant methods, etc., as known in the art. Alternatively complex antigens may be used, for example cell lysates, virus which may be inactivated, bacterial cells or fractions derived therefrom, and the like.

The methods of the invention are useful when used in conjunction with vaccines such as, but not limited to, those for treating chronic bacterial infections, e.g. tuberculosis, etc.; chronic viral infections such as those associated with herpesvirus, lentivirus and retrovirus, etc. Antigens of interest may also include allergens, e.g. for the conversion of a Th2 to a Th1 type response.

Potential tumor antigens for immunotherapy include tumor specific antigens, e.g. immunoglobulin idiotypes and T cell antigen receptors; oncogenes, such as p21/ras, p53, p210/bcr-abl fusion product; etc.; developmental antigens, e.g. MART-1/Melan A; MAGE-1, MAGE-3; GAGE family; telomerase; etc.; viral antigens, e.g. human papilloma virus, Epstein Barr virus, etc.; tissue specific self-antigens, e.g. tyrosinase; gp100; prostatic acid phosphatase, prostate specific antigen, prostate specific membrane antigen; thyroglobulin, .alpha.-fetoprotein; etc.; and over-expressed self antigens, e.g. her-2/neu; carcinoembryonic antigen, muc-1, and the like.

Tumor cell derived protein extracts or RNA may be used as a source of antigen, in order to provide multiple antigens and increase the probability of inducing immunity to more than one tumor associated antigen. Although the target antigens are initially undefined, the immunogen can be later identified.

Antigenic formulations will typically contain from about 0.1 μg to 1000 μg, more preferably 1 μg to 100 μg, of the selected antigen. The antigen composition may additionally contain biological buffers, excipients, preservatives, and the like.

The antigen is administered to the host in the manner conventional for the particular immunogen, generally as a single unit dose of an antigen in buffered saline, combined with the adjuvant formulation, where booster doses, typically one to several weeks later, may additionally be delivered enterally or parenterally, e.g., subcutaneously, intramuscularly, intradermally, intravenously, intraarterially, intraperitoneally, intranasally, orally, etc. Subcutaneous or intramuscular injection is, however, preferred.

Receptors for cystatin-like chemoattractants may represent selective targets for therapeutic treatment of particular blood leukemias. For example, CD56+CD4+ hematodermic neoplasms were recently characterized as being oncogenically transformed plasmacytoid DC. This rare, aggressive malignancy is characterized in part by significant skin involvement, which is often times the first clinical symptom of disease. Given the high levels of chemerin RNA message in the skin, and expression of the skin homing receptor cutaneous lymphocyte antigen (CLA), CMKLR1:chemerin interactions may contribute to the cutaneous tropism observed in pDC leukemia. Given its restrictive expression, CMKLR1 or other receptors for cystatin-like chemoattractants may represent a selective target for ablative therapy for pDC leukemias, as has been demonstrated for chemokine receptor CCR4 and T cell leukemia and lymphoma (example reference in email). Agonists or antagonists of CMKLR1 or other receptors for cystatin-like chemoattractants may also be used to limit tumor metastasis (such as the skin metastases observed in pDC leukemia), as demonstrated for chemokine receptor CXCR4 (example reference in email).

### Pharmaceutical Formulations

Formulations of cystatin-like chemoattractant agents, e.g. specific binding members including antibodies and other ligands; small molecules that bind and/or inhibit and/or mimic activity; and the like, may be administered to a patients, e.g. in a form stabilized for stability and retention in the targeted region. The formulation may comprise one, two or more therapeutic agents, and may further comprise other agents, e.g. antigens for increased immune response.

Strategies for increasing retention include the entrapment of the agent in a biodegradable or bioerodible implant, preferably the implant is comprised of a non-immunogenic material. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed include organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g., intravascular (intravenous, intraarterial, intracapillary) administration. The effective amount of a therapeutic composition to be given to a particular patient will depend on a variety of factors, several of which will be different from patient to patient. A competent clinician will be able to determine an effective amount of a therapeutic agent. The compositions can be administered to the subject in a series of more than one administration. For therapeutic compositions, regular periodic administration (e.g., every 2-3 days) will sometimes be required, or may be desirable to reduce toxicity. For therapeutic compositions that will be utilized in repeated-dose regimens, antibody moieties that do not provoke immune responses are preferred.

Those of skill will readily appreciate that dose levels can vary as a function of the specific compound, the severity of the symptoms and the susceptibility of the subject to side effects. Some of the specific complexes are more potent than others. Preferred dosages for a given agent are readily determinable by those of skill in the art by a variety of means. A preferred means is to measure the physiological potency of a given compound.

### EXPERIMENTAL

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which scope will be determined by the language in the claims.

It must be noted that as used herein and in the appended claims, the singular forms “a”, “and”, and “the” include plural referents unless the context clearly dictates otherwise. Thus, for example, reference to “a mouse” includes a plurality of such mice and reference to “the cytokine” includes reference to one or more cytokines and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All publications mentioned herein are incorporated herein by reference for all relevant purposes, e.g., the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### Example 1

### CMKLR1 Expression and Chemerin-directed Chemotaxis Distinguish Plasmacytoid from Myeloid Dendritic Cells in Human Blood

Materials and Methods

Antibodies and Reagents. Anti-CD3, -CD11c, -CD14, -CD16, -CD19, -CD20, -CD56, —CD83, -CD123, -HLADR dye-linked mAbs, purified HLADR, BDCA2, BDCA4, and secondary α-mouse APC and α-rat PE for immunofluorescence studies were obtained from BD PharMingen, Miltenyi, eBioscience, Jackson Labs, and Caltag. CXCL12, CCL2, CCL19, CCL21, IL4, GM-CSF were purchased from R&D Systems, LPS was purchased from Sigma, CMFDA was purchased from Molecular Probes, and phosphothioated CpG oligonucleotides were purchased from Operon.

Mammalian Expression Vector Construction and Generation of Stable Cell Lines. The coding regions of huCMKLR1 and mCMKLR1 were amplified from genomic DNA with an engineered N-terminal HA tag, and cloned into pcDNA3 (Invitrogen). The full-length chemerin cDNA encoding chemerin was amplified from human liver RNA (BD Clontech) and engineered to have an N-terminal 6×His tag after the signal sequence and cloned into pcDNA3. Transfectants of huCMKLR1, mCMKLR1, chemerin or empty vector were generated and stable lines selected in the murine pre-B lymphoma cell line L1.2 essentially as described. Transfected cells were in some cases treated with 5 mM n-butyric acid for 24 h before experimentation.

Chromatography and LC/MS/MS. 1.6 L of human serum (Serologicals) was filtered and used as starting material. Heparin sepharose (Amersham) and SP sepharose cation exchange (Amersham) chromatography were performed using 50 ml and 2 ml columns and a low-pressure peristaltic pump (Masterflex). Single bed volumes of 0.1 M stepwise increments of NaCl buffer (in 50 mM MES, pH 6.3) was used to elute proteins off the column. Columns were washed twice with 0.1 M NaCl buffer before salt increments were started. Gel filtration FPLC (Superdex75, Amersham) was performed, and 250 ul fractions were collected. Following all chromatography steps, protein concentration was determined in each fraction by BCA (Pierce), protein eluants were assayed in transwell migration with CMKLR1/L1.2 transfectants, and active fractions were pooled, diluted as appropriate, and applied to subsequent separation columns. The purified protein was separated by SDS-PAGE, and the bands were analyzed by LC/MS/MS (Protein Chemistry Core Facility, Columbia University, NY). The tryptic mass values were used in a Mascot search of public peptide databases. Acrylamide adducts (cysteine modifications) were taken into consideration when mass values were searched.

Bacterial Production of Recombinant Chemerin. The coding region of the predicted secreted form of chemerin was amplified by RT-PCR from human liver RNA (BD Clontech) and directionally cloned into the EcoRI/HindIII sites of pET42a (Novagen), in-frame with upstream GST and 6×His tags. Following IPTG induction in E. coli strain BL21, inclusion bodies were harvested, and the fusion protein was solubilized in 6M guanidine HCl and refolded by dropwise dilution with refolding buffer (0.1M Tris HCl pH 8.0, 1 mM oxidized glutathione, and 0.1 mM reduced glutathione) to final protein concentrations of 10-100 ug/ml.

RNA Expression Analysis. Dot blot RNA arrays were purchased from BD Clontech and hybridizations were performed according to the manufacturer's recommendation. A full-length gel-purified chemerin cDNA probe were radiolabeled with ³²P using RediPrime reagents (Amersham) according to manufacturer's specifications. RT-PCR expression analysis of chemerin was performed using 500 ng total RNA (purchased from BD Clontech) as cDNA synthesis template. Full-length chemerin was amplified using intron-spanning primers, and G3PDH primers that spanned intron H were used. “No RT” controls were negative for chemerin amplicons.

Harvesting PBMC and Generating Cultured Monocyte-derived DC. The Institutional Review Board at Stanford University approved all human subject protocols, and informed consent was obtained for all donations. Plasma was collected from blood samples drawn into tubes containing heparin, EDTA, or Na citrate (BD Vacutainer). Human blood was collected and peripheral blood mononuclear cells (PBMC) were harvested following Histopaque 1077 gradient separation. Miltenyi MACs magnetic bead CD14+ separation was performed according to manufacturer's specifications. CD14+ monocytes were cultured in RPMI+10% FCS+additives at 2-10 million cells/ml with 100 ng/ml GM-CSF and 100 ng/ml IL-4 for 7 days to generate immature DC. In some cases, the DC were cultured an additional 24 hr with 10 ng/ml LPS to generate mature (activated) DC.

Cell Sorting and Wright Giemsa Stain. Leukocytes were stained as described and sorted by standard flow cytometric techniques (FACsvantage, Stanford University Digestive Disease Center Core Facility). Between 1-10×10³ sorted cells were loaded into cytospin chambers and centrifuged onto glass slides. The slides were stained with Wright-Giemsa dye by standard automated techniques at the VA Hospital Hematology Lab (Palo Alto) and examined by light microscopy with a 40× objective.

In vitro Transwell/transendothelial Chemotaxis. 5-um pore Transwell inserts (Costar) were used. For transendothelial migration, Transwell inserts were coated with gelatin, seeded with 10⁵ human umbilical vein endothelial cells (HUVEC) (passage <8), and incubated o/n, as previously described. Monolayers were rinsed with chemotaxis media before use. Chemotaxis media consisted of RPMI+10% FCS+ additives, and 100 μl cells were added to the top well, and test samples were added to the bottom well in a 600 μl volume. Migration was assayed for 2-5 hr at 37° C., then the inserts were removed, and the cells that had migrated through the filter to the lower chamber were in some cases stained and counted by flow cytometry. An equivalent number of beads was added to each tube to allow the cell count to be normalized. A ratio was generated and percent input migration is displayed. For in vitro cultured DC migration, 1-6×10⁵ cells were added to the top well and migrated cells were counted using a DC cell gate based on forward and side scatter. For human primary blood cell migration, cells were pre-incubated 1-3 hr in media to allow for recovery of receptor expression. 1×10⁶ PBMC were added to the top well, and migrated cells were stained (Lin FITC, HLADR PE, CD123 Cychrome, CD11c APC) and analyzed by 4-color flow cytometry. For some donors, incubation times resulting in optimal recovery of chemerin functional response was determined empirically and used. For transfectant migration, ˜2.5×10⁵ cells/well were used, and the number of cells counted in 30 s was used as the migration output. The results are reported as % input migration. Either a predetermined volume of chemerin CM eliciting >30% CMKLR1/L1.2 transfectant migration (along with an equivalent volume of empty vector (pcDNA3) L1.2 transfectant CM as a negative control), or refolded recombinant E. coli-expressed chemerin was used. Student's t-test (two-tailed with unequal variance) was used to determine statistical significance.

Anti-CMKLR1 mAb. The immunizing amino-terminal CMKLR1 peptide was synthesized by Stanford PAN facility and conjugated to KLH according to the manufacturer's specifications (Pierce). CFA and IFA were purchased form Sigma. Wistar Furth rats were purchased from Charles River. An ELISA-based assay (BD Pharmingen) was used to determine the isotype of our rat anti-human CMKLR1 mAb.

Results

A CMKLR1-specific mAb Stains Culture-derived DC. We generated a monoclonal antibody designated BZ332 (IgG₂ₐκ) to human CMKLR1 after immunizing rats with a KLH conjugate of an amino-terminal CMKLR1 peptide comprised of residues 10-24 and having the sequence NH₂-TSISYGDEYPDYLDSIWLEDLSPLC-COOH. Hybridomas producing anti-huCMKLR1 mAbs were subcloned, and specificity was confirmed by reactivity with human but not mouse CMKLR1 transfectants, and by lack of reactivity with L1.2 cells expressing human CCR9 and CCR10. Mouse CMKLR1 shares 80% amino acid identity and is more homologous to human CMKLR1 than any human protein, and thus represents the most probable candidate for mAb cross-reactivity, which was not observed. Reactivity with CXCR1-through-6 and CCR1-through-10 was excluded by lack of staining of blood cell subsets or cultured human cells known to express these receptors.

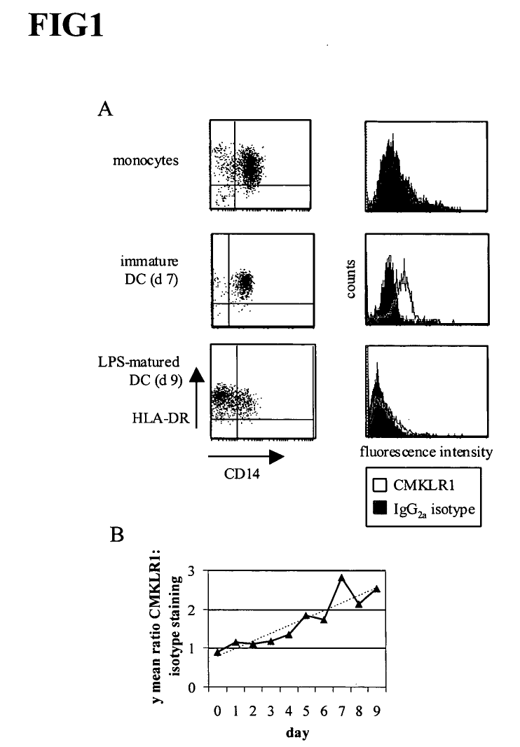

We used mAb BZ332 to assess expression of CMKLR1 by dendritic cells. We found that in vitro cultured, monocyte-derived immature DC generated with IL4 and GM-CSF expressed CMKLR1, whereas precursor monocytes or LPS-matured DC did not express the receptor (FIG. 1). Monocyte derived DC have been considered to be a model of mDC observed in vivo, suggesting involvement of the receptor in mDC function. Since culture models of specialized leukocytes may fail to recapitulate the phenotypic characteristics of physiologic cell subsets in vivo, however, we asked whether CMKLR1 is expressed by circulating mDC, where it might influence their recruitment from the blood.

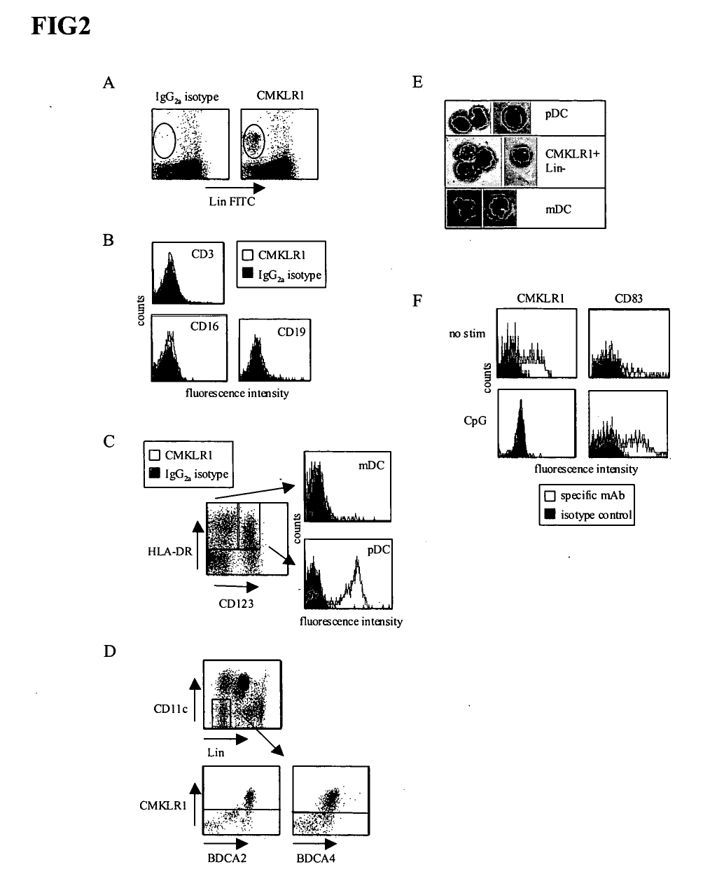

Expression of CMKLR1 by Circulating Plasmacytoid but not Myeloid DC. Blood DC make up ˜1% of circulating peripheral blood mononuclear cells (PBMC), and are characterized as Lin− MHC class II+ (CD3-CD14-CD16-CD19-CD20-CD56-HLADR+). Immuno-fluorescence staining of total PBMC revealed CMKLR1 expression limited to a small subset of lineage marker negative cells, consistent with expression by a subset of circulating DC (FIG. 2A). Lin+ cells were negative for mAb reactivity, suggesting that the receptor is not expressed detectably by circulating lymphocyte subsets (FIG. 2A). As shown in FIG. 2B, this was confirmed by gating on CD3+ T cells, CD19+ B cells, CD16+ NK cells. In addition, circulating monocytes among the Histopaque-isolated PBMC were also negative (FIG. 1A). Peripheral blood DC can be further subdivided by expression of CD123: myeloid DC are CD123−, whereas plasmacytoid DC are CD123+. Surprisingly, CMKLR1 was not expressed by Lin-HLADR+CD123− mDC, whereas CD123+ pDC were uniformly positive (FIG. 2C). Blood CD123+CD11c−CMKLR1+ cells co-express the pDC-specific markers BDCA2 and BDCA4 as well, confirming the staining of pDC in blood (FIG. 2D).

To confirm our immunophenotyping results, we sorted CMKLR1+Lin− blood mononuclear cells for Wright-Giemsa staining to examine the morphology of the cells. We also sorted and stained blood pDC (Lin-HLADR+CD123+CD11c−) and mDC (Lin-HLADR+CD123− CD11c+) for comparison. As predicted, sorted CMKLR1+ cells and pDC shared similar morphology, consistent with descriptions of pDC appearance in the literature (FIG. 2E). CMKLR1+ cells and pDC were round, smooth cells with generally circular nuclei and pale peri-nuclear regions. Sorted mDC display a clearly different morphology, with multiple cytoplasmic projections and lobulated, protruding nuclei. Thus, both traditional morphologic and immunophenotypic analysis indicates selective expression of CMKLR1 on pDC versus mDC.

DC alter their chemoattractant receptor expression profiles upon stimulation with toll receptor or costimulatory receptor ligands. pDC activated by overnight incubation with CpG oligonucleotides (or CD40L+IL3) down-regulated CMKLR1 receptor expression (FIG. 2F).

The selective expression of CMKLR1 by immature plasmacytoid DC in blood was surprising in light of the observed expression of CMKLR1 on monocyte-derived DC in vitro. However, the culture-derived cells may present an a typical or specialized DC phenotype, sharing some features of both myeloid (e.g. CD11c) and plasmacytoid DC differentiation. (In fact, these monocyte-derived DC also express another antigen that is specific for pDC vs. mDC in blood, BDCA4). It is known that DC of different phenotypes can be generated based on specific combinations of cytokines present during their in vitro derivation, and it will be of interest in future studies to assess the factors responsible for regulation of CMKLR1, as well as those regulating other markers of physiologic pDC differentiation.

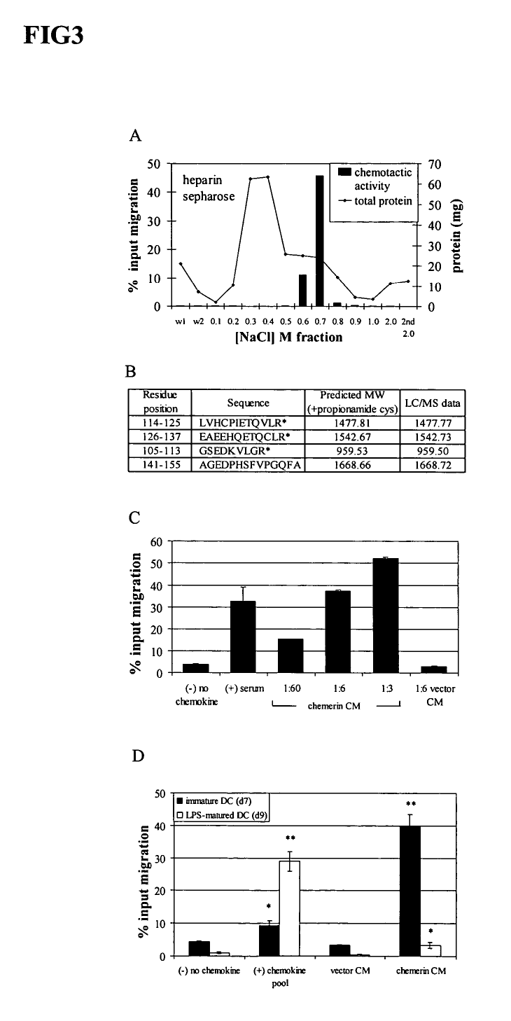

Identification of a Potent Serum CMKLR1-dependent Chemoattractant as Chemerin. We used CMKLR1 transfected L1.2 cell chemotaxis to detect and isolate a natural ligand for the receptor from human serum. Briefly, 1.6 L of human serum (128 g total protein) was filtered and applied to a heparin sepharose column, and fractions were eluted using stepwise increments of NaCl buffer (FIG. 3A). The 0.7 M NaCl fraction contained the bulk of activity, and was highly enriched in chemoattractant protein, as >99.9% of serum proteins were eliminated. The separation and protein identification proceeded via cation exchange and gel filtration column chromatography, SDS polyacrylamide gel electrophoresis, and liquid chromatography/tandem mass spectrometry (LC/MS/MS) (data not shown). Consistent with the recent reports describing a CMKLR1 ligand from human ascites or hemofiltrate, four mass values from a tryptic digest of the protein confirmed the identity of the active chemotactic agent in serum as the protein product of the tazarotene-induced gene 2 (chemerin, or chemerin, FIG. 3B). Conditioned media from chemerin transfected cells acted as a chemoattractant for CMKLR1 transfectants (FIG. 3C), and for immature monocyte-derived DC (FIG. 3D).

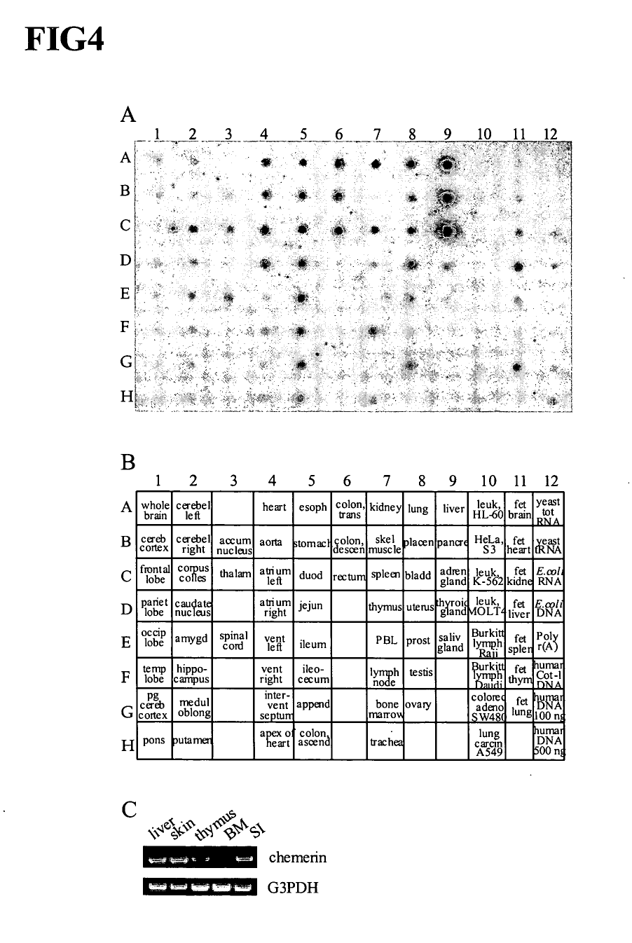

Chemerin RNA is expressed at readily detectable levels in numerous tissues and organs (FIG. 4). For example, chemerin message is found in the lymph nodes, where it may contribute to pDC homing to secondary lymphoid tissues. Although most tissues, except components of the nervous system, seem to express significant levels, the most abundant sources of chemerin mRNA appear to be liver, pancreas, and adrenal gland. High level expression by the liver may be responsible for the high levels of chemerin in the serum. Chemerin message is also abundant in the skin (FIG. 4C).

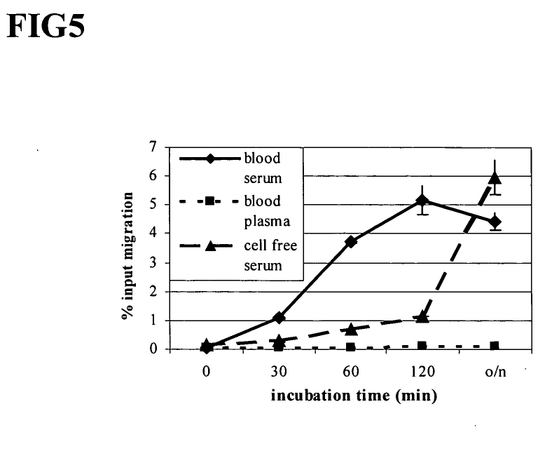

In our experiments leading to the identification of chemerin as a dominant serum chemoattractant for CMKLR1, we observed that, in contrast to serum, human plasma displayed very little attractant activity. We hypothesized that factors activated upon blood clotting were responsible for the increased chemerin activity, and since coagulation and fibrinolysis are enzymatic and thus time dependent processes, we compared chemerin activity in either serum or plasma from normal or anti-coagulated blood from the same donor over time. We found that serum displays significantly more chemoattractant activity than plasma over matched time intervals (FIG. 5). To determine if the increase in chemerin activity was dependent on the presence of blood cells, we collected cell-free fluid from blood centrifuged immediately after blood draw (from the same donor) and assayed the samples for chemerin activity. Chemerin activity in “cell free serum” also increased over time, although with somewhat delayed kinetics (FIG. 5). We conclude that chemerin circulates in a less or inactive proform in blood and that factors associated with or induced by the clotting or fibrinolytic cascades can activate chemerin in plasma. Furthermore, our results are consistent with previous studies demonstrating a role for cellular factors in the proteolytic activation of chemerin, since the presence of blood cells accelerates chemerin activation during blood coagulation.

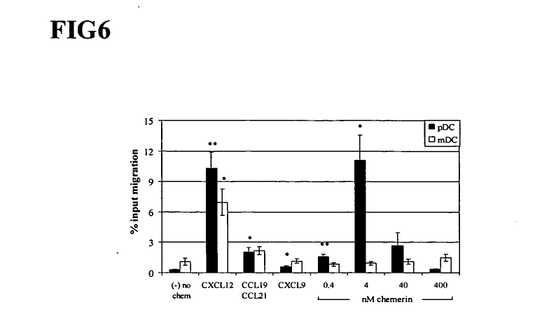

Chemerin Attracts Blood Plasmacytoid but not Myeloid DC. We evaluated the ability of the ligand to attract blood DC in assays of cell migration across monolayers of human umbilical vein endothelium, pDC migrated significantly to recombinant attractant in a dose dependent manner (in transendothelial migration assays, mean 11.1±2.5% S.E. input migration to 4.0 nM recombinant bacterial-expressed chemerin vs. 0.3±0.1% background migration (n=3 different donors; p<0.05), FIG. 6). The full-length recombinant chemerin was highly active in these assays, likely reflecting spontaneous processing to the active form by the endothelial cells or blood leukocytes in the assay system. pDC also migrated well to the general leukocyte attractant CXCL12 (10.3±1.6%), and significantly but less well to the CCR7 ligands CCL19 (ELC) and CCL21 (SLC) (2.0±0.5%, p<0.05 vs. media control). In contrast mDC did not migrate above background to chemerin at any concentration tested (0.9±0.2% for 4.0 nM chemerin vs. 1.1±0.4% background migration). Although pDC migration across bare transwell membranes is less efficient overall, chemerin in conditioned medium also attracted pDC in standard transwell chemotaxis assays (mean 6.0±1.0% S.E. input migration to conditioned media from chemerin-transfected L1.2 cells vs. 1.6±0.3% input migration to control vector transfected medium (n=13 different donors; p<0.0005)). Thus chemerin is a potent attractant for circulating pDC but not for blood mDC, correlating with the differential expression of its receptor.

We have found that the chemoattractant receptor CMKLR1 is expressed by plasmacytoid DC in blood, distinguishing pDC from naive and memory lymphocytes, monocytes, granulocytes and even blood myeloid DC. CMKLR1 confers on circulating pDC the ability to respond to a unique chemoattractant, chemerin. Chemerin is widely expressed at the RNA level, and the translated protein is found in abundance in blood. Our data suggest that the less or inactive proform of chemerin is present in plasma, and it is converted into a potent pDC chemoattractant following blood coagulation. These results support a potential mechanism for the recruitment of pDC to sites of bleeding, and for bridging hemostasis with the innate and adaptive immune responses following tissue damage.

The chemerin-encoding gene chemerin was initially cloned by differential display as being upregulated in in vitro cultured human skin rafts treated with the anti-inflammatory retinoid tazarotene. It was also shown to be upregulated in a hormone-treated, osteoclastogenic mouse bone marrow stromal cell line ST2. Indeed, we found that conditioned media from the ST2 cell line treated with 1.25 dihydroxyvitamin D3 and dexamethasone was chemotactic for CMKLR1 transfectants. These results, together with our RNA expression data, suggest that chemerin message is constitutively expressed in a number of tissues, and that it can be regulated as well, particularly in response to retinoid and steroid receptor stimulation.

It is clear, however, that post-translational modification of chemerin in the form of proteolytic processing also regulates its chemoattractant activity. Recombinant full-length chemerin effects CMKLR1-signaling only when presented at high concentrations compared to fully active forms. Structural analyses show that the attractant is activated by proteolysis and release of short carboxy-terminal peptides. Interestingly, chemerin is spontaneously activated by co-culture with cells, and by factors in supernatants of cultured cells (see for example the migration of CMKLR1 transfectants to conditioned media from chemerin-expressing cells in FIG. 3C). This explains the potent activity of recombinant chemerin in our studies of transendothelial cell pDC chemotaxis, since in this setting the endothelial cells, or the migrating cells themselves, can spontaneously activate the recombinant attractant.

Our results suggest that proteases activated during the coagulation or fibrinolytic cascades may also, directly or indirectly, lead to carboxy-terminal cleavage and subsequent chemerin activation. Of course, inflammation is also associated with the activation of coagulation/fibrinolytic enzymes, as shown in allergic contact dermatitis and delayed-type hypersensitivity lesions, and synovial fluid from spondyloarthropathic or rheumatic joints. Thus, the hemostatic systems that trigger chemerin activation during blood clotting may also participate, along with other inflammatory protease cascades, in regulating pDC recruitment to sites of inflammation.

In addition to its role as a chemoattractant receptor, CMKLR1 is a demonstrated co-receptor for primary HIV-1 strain 92UG024. In this context, our finding of selective expression of CMKLR1 by pDC suggests a potential explanation for recent data that plasmacytoid DC can be infected more easily than mDC by certain HIV-1 strains. pDC may be efficient targets for HIV infection because they express CD4, they are present in blood and secondary lymphoid organs, and they express co-receptors such as CXCR4, CCR5 and now CMKLR1. IFNα is known to interfere with productive HIV infection, and since pDC are the primary IFNα producing cell in the body, targeting and eliminating pDC may be important for productive and stable HIV-1 infection. Multiple co-receptor blockade, including agents directed against CMKLR1, may be a useful therapeutic approach to controlling HIV-1 in infected patients.

In conclusion, our findings demonstrate that CMKLR1 is a key mediator of pDC recruitment from the blood into tissue sites enriched in active chemerin. The enhanced activity of chemerin in response to blood clotting and to cellular protease activators may render it uniquely suited to recruit pDC to sites of bleeding, tissue damage, and inflammation. pDC, through alpha interferon production and antigen processing, are thought to be important in bridging the innate and adaptive immune responses: rapid recruitment to sites of inflammatory protease activation may be critical to this role.

The down-regulation of CMKLR1 upon DC activation and maturation, along with enhanced responsiveness to CCR7 ligands, is consistent with the extensive reprogramming of DC migration during the immune response to pathogens, and may help permit their migration as APC to lymph nodes via lymphatics. The significance of selective CMKLR1 expression to the unique sensitivity of pDC to HIV-1 infection is now amenable to study as well. The identification of this pDC selective chemoattractant and receptor may offer opportunities to regulate the migration of these versatile and potent cells, either to enhance anti-viral responses or to modulate immune activity.

### Example 2

### Serine Proteases of the Coagulation Cascade Activate Chemerin

Materials and Methods

Plasma, Serum, and Serine Proteases. Serum was stripped of heparin binding proteins (including chemerin) by collecting the “flow-through” after passage over a heparin sepharose column. Plasma was collected from blood samples drawn into tubes containing heparin, EDTA, or Na citrate (BD Vacutainer). An equivalent amount of anticoagulant was added to each “serum” sample before testing to control for the anticoagulant present in the “plasma” samples. An amount of E. coli-expressed chemerin showing less than 5% input migration was incubated with an equivalent volume of serum or plasma for 5 min at 37° C. and then tested in chemotaxis with CMKLR1/L1.2 transfectants. Protease inhibitors (aprotinin (0.16 mg/ml) and E64 (1.67 mg/ml) (Sigma) were pre-incubated with serum samples for 1 hr before chemerin was added. Various concentrations of serine proteases were incubated with chemerin for 5 min at 37° C. and then tested in chemotaxis. In each case, digestion was arrested by placing the tubes on ice and immediately diluting 1:50 into cold chemotaxis medium for assay. Physiologic concentrations of blood coagulation zymogens (all in ug/ml) are as follows: thrombin 100 (Sigma), factor X 10 (Pierce), factor VII 0.5, factor IX 5, factor XI 5, factor XII 30, kallikrein 40 (Enzyme Research Laboratories), plasmin 200 (Sigma), tPA 0.005 (Calbiochem), uPA 0.008 (American Diagnostica).

Results

Serum, but Not Plasma, is Chemotactic for CMKLR1. In striking contrast to the potent attractant activity observed with serum, freshly isolated plasma demonstrated little activity. We observed that overtime as blood clotting progressed, there was an increase in chemerin activity as compared with anti-coagulated plasma. It was clear from our experiments that chemerin was proteolytically processed, and our observations that serum possessed more activity than plasma led us to the hypothesis that proteases capable of processing and activating chemerin were triggered in response to blood clotting. Chemerin may circulate in its pro-form, and become activated by proteolysis triggered during blood clotting. As coagulation and subsequent clot lysis are mediated by a cascade of serine proteases, we examined chemerin for potential cleavage sites and identified a canonical serine protease cleavage site five amino acids from the carboxy-terminus, NH2 . . . FAFSK|ALPRS-COOH.

Serum Enzymes Activate Chemerin. To directly test the ability of proteolytic substances to activate chemerin, we needed a source of full-length chemoattractant, as it was clear that our mammalian-expressed form was being modified at some point during its production. We therefore turned to bacterially-expressed recombinant chemerin, which was shown by mass spectrometric analysis to be full-length. We used two versions of recombinant chemerin, either with an N-term GST fusion and an endogenous C-term, or an endogenous N-term and a 6×His tagged C-term (N-term refers to the secreted form, not including the signal sequence), both of which displayed equivalent activities. To directly assess the enzyme activity in serum, we first needed to remove endogenously activated chemerin, and thus we prepared a “stripped” serum, in which endogenous heparin-binding proteins (including chemerin) were removed by column chromatography.

Using recombinant chemerin at a concentration showing minimal initial activity (titred to yield only 5% migration of CMKLR1 transfectants), we found that incubation with “stripped” serum dramatically enhanced chemotactic activity for CMKLR1 (FIG. 5.2). The triggering event was rapid, as maximal chemerin chemotactic activity was reached within 5 minutes of incubation at 37° C. Even prolonged, one-hour incubation with plasmin failed to degrade chemerin to inactivity, whereas such treatment with trypsin destroyed the ability of chemerin to attract CMKLR1-expressing cells (data not shown). We conclude that blood coagulation activates or releases a factor(s) that can rapidly trigger activation of chemerin, providing a mechanism for rapid pDC recruitment to sites of vascular injury or tissue damage.

Serine Proteases of Coagulation/Fibrinolysis Activate Chemerin. To further dissect the mechanism of chemerin activation, we found that treatment of serum with the general serine protease inhibitor aprotinin (but not the cysteine protease inhibitor E64) was able to block the serum activation of chemerin. This result indicates that serine proteases activated upon blood clotting are likely activating chemerin. To test the general ability of serine proteases to activate chemerin, we observed that limited trypsin proteolysis of recombinant chemerin resulted in an increase in chemotactic activity, in a protease-dose-dependent fashion. To further determine which serine protease(s) of the secondary hemostatic system might be involved in activating the chemoattractant, recombinant chemerin was incubated with factor VIIa, IXa, Xa, XIa, XIIa, kallikrein, thrombin, and plasmin for 5 min and assayed for chemotactic activity. Enhanced chemotactic activity of chemerin was most effectively and efficiently generated by plasmin, an abundant blood and tissue serine protease that cleaves fibrin and leads to clot lysis. Even at concentrations 10× lower than physiologic blood plasminogen concentrations, plasmin was able to activate chemerin more efficiently than serum. Factor XIIa was also quite potent, showing activity similar to that of serum when used at physiologic blood levels. Other serine proteases of the coagulation cascade may also be able to contribute to chemerin activity, particularly in a setting where they are concentrated.

Plasmin is generated by cleavage of plasminogen by either serine protease plasminogen activators uPA or tPA. Both tPA and uPA were able to activate chemerin, although the required enzyme concentrations were much higher (˜1000 fold) than their observed plasma zymogen concentrations. Both plasminogen activators display increased abilities to activate plasminogen when in the bound state, particularly tPA which displays a kinetic acceleration of ˜50-fold in plasminogen activation in the presence of fibrin. Thus their low plasma concentrations do not adequately reflect their effective physiologic concentrations. Furthermore, uPA concentrations in the range we used effectively cleave its primary physiologic target, plasminogen.

We found that activation of chemerin by plasmin resulted in a ˜20-fold increase in chemoattractant potency (FIG. 5.5). Both forms of the chemoattractant, however, were observed to induce robust migration, and it is interesting to note that the amplitude of responses is roughly equal. Both forms also induced a dose-dependent bell-shaped chemotactic response, which is commonly observed in chemokine-induced migration. This effect was not observed in receptor-ligand binding studies, and implies that receptor desensitization may play a role in the biology of CMKLR1-mediated recruitment. The direct cleavage of chemerin by plasmin can be observed by polyacrylamide gel electrophoresis. Mass spectroscopic analysis of plasmin-treated chemerin revealed the cleavage site to be NH2 . . . FAFSK|ALPRS-COOH, as we had predicted. A similar shift in size was observed by polyacrylamide gel electrophoresis when serum was used as the proteolytic agent.

Preliminary mass spectroscopic analysis of the serum cleavage product indicates a truncated C-term, as well as evidence of amino-terminal processing. The C-term site, NH2 . . . FPGQFAFS|KALPRS-COOH is not consistent with the solo activity of serine proteases. Given that other C-termini have been reported (NH2 . . . FPGQF|AFSKALPRS-COOH), we predict that additional proteolytic enzymes, such as the abundant serum carboxypeptidase CPN may play a role in chemerin processing. The role of coagulation and fibrinolytic pathway enzymes in chemerin activation likely represents a necessary component of a more elaborate regulatory process.

The extracellular proteolytic processing of a number of chemokines has been shown to modulate their chemotactic activities. For example, CXCL12 cleavage by membrane bound protease CD26 (dipeptidylpeptidase IV) generates a CXCR4 antagonist. CD26 cleavage of CCL5 (RANTES) reduces its activity to attract CCR1-expressing cells. Gelatinase A cleavage of CCL7 (MCP3) generates a CCR5 antagonist. Basic platelet protein is cleaved by cathepsin G to generate CXCL7 (NAP2), a potent neutrophil chemoattractant that acts through CXCR2 (112). The extracellular processing of chemoattractants represents a potentially critical regulatory mechanism for the physiologic recruitment of leukocytes.

Plasmin-mediated proteolysis has been shown to activate the pleiotropic cytokine TGF-β as well as the abundant, pro-inflammatory plasma chemokine CCL14 (HCC-1 or hemofiltrate CC chemokine 1), which attracts T cells, eosinophils, and monocytes. Chemerin thus joins a growing family of immuno-modulatory proteins regulated by fibrinolytic enzymes, and directly couples pDC recruitment to the vascular and tissue inflammatory response. Interestingly, the CMKLR1-related subfamily of leukocyte GPCRs also includes chemoattractant receptors for complement components C5a and C3a, blood proteins that, like chemerin, are proteolytically activated and involved in rapid responses linking innate and adaptive immunity. FPRL1, another member of the receptor subfamily, is expressed on neutrophils and acts as a chemoattractant receptor for human cathelicidin LL-37, which is also subject to proteolytic processing. A hallmark of this chemoattractant receptor subfamily thus appears to be an affinity for rapidly activatable ligands involved in host defense.

### Example 3

### Monocytic Mouse Leukocytes Express CMKLR1 and Migrate to Chemerin

Materials and Methods

Anti-Mouse Antibodies and Reagents. Anti-CD3, -CD11c, -CD11b, -CD14, -CD19, -B220, -DX5, -Gr1, -IA/E class II, -Ly6C, -TCRβ dye-linked mAbs, purified Fc block (mouse anti-mouse CD16/32), and secondary α-rat PE (human and mouse adsorbed) for immunofluorescence studies were obtained from BD PharMingen, Miltenyi, eBioscience, Jackson Labs, and Caltag.

Transwell chemotaxis. Performed and displayed as previously described. For mouse primary blood cell migration, ˜2.5×10⁵ cells/well were used. Cells were pre-incubated 1 hr in media to allow for recovery of receptor expression. Migrated cells were stained with F4/80 FITC and CD11 b APC and analyzed by flow cytometry.

Harvesting Mouse Leukocytes. Balb/c, 129S, C57BL/6, and JHD mice were obtained from Taconic. To harvest blood leukocytes, mice were given a fatal dose of anesthesia (ketamine/xylazine) as well as an i.p. injection of heparin. Mouse blood was collected by cardiac puncture and/or aspiration of pooled blood from chest cavity. Up to 1 ml of blood was added to 5 ml 2 mM EDTA in PBS, and then 6 ml of 2% dextran T500 (Sigma) was added to crosslink RBC. The mixture was incubated for 1 hr at 37° C., the supernatant was removed and pelleted, and the cells were resuspended in 5 ml RBC lysis buffer and incubated at RT for 5 min. The cells were pelleted, and resuspended for use in cell staining or chemotaxis. Lymph node, thymus, and spleen cells were harvested by crushing the organs over wire mesh, and in the case of splenocytes, performing RBC lysis. Bone marrow cells were harvested by flushing femurs and tibias with media and performing RBC lysis. Peritoneal lavage cells were harvested by injecting 10 ml of PBS i.p. and collecting the exudate.

Anti-mCMKLR1 mAb. The immunizing amino-terminal mCMKLR1 peptide (residues 10-24 in the amino terminus of mCMKLR1 having the sequence NH₂-DSGIYDDEYSDGFGYFVDLEEASPWC-COOH) was synthesized by Stanford PAN facility and conjugated to KLH according to the manufacturer's specifications (Pierce). CFA and IFA were purchased form Sigma, and immunizations were performed as described for the human CMKLR1 peptide. Wistar Furth rats were purchased from Charles River. An ELISA-based assay (BD Pharmingen) was used to determine the isotype of our rat anti-mouse CMKLR1 mAb.

Results

Identifying CMKLR1+ Leukocytes In the Mouse. In initial flow cytometry experiments, we were unable to identify a population of mouse blood leukocytes that expressed the CMKLR1 receptor, based on typical lymphocyte/monocyte forward and side light scatter gates. When we analyzed un-gated cells however, we noted a significant population positive for receptor expression. Gating on these CMKLR1+ cells, and then displaying these cells on a forward and side scatter plot via back-gating, a population of large, granular CMKLR1+ cells was revealed. The scatter profile of these CMKLR1+ cells was generally consistent from strain to strain. Setting a forward and side scatter gate in favor of these large, granular cells revealed uniform CMKLR1 staining. These cells represented between 1-5% total leukocytes obtained following dextran-RBC depletion and RBC lysis.

Like human pDC, mouse pDC are rare cells, making up less than 0.5% of total blood leukocytes. Mouse pDC are identified by expression of B220, CD11c, Ly6C, and an absence of the T, B, and NK lymphocyte markers CD3, CD19, and DX5. B220+CD11c+Ly6C+Lin− pDC did not express the receptor.

We used flow cytometry to investigate the immunophenotype of CMKLR1+ cells. A gate based on CMKLR1+ cell staining was set, and a panel of antibodies and isotype controls were employed to profile the leukocyte subset. CMKLR1+ cells expressed high levels of CD11b, CD14, and F4/80, all markers of monocytic-lineage cells (FIG. 6.4). The CMKLR1+ cells were negative for lymphocyte markers (B cell markers CD19 and B220, T cell markers CD3 and TCRβ, NK marker DX5), expressed low levels of the granulocyte marker Gr-1, and displayed variable low-to-no levels of MHC class II and CD11c. Based on these flow cytometry results, it is unlikely that the CMKLR1+ cells are typically defined mouse monocytes. Circulating mouse monocytes display a light scatter profile slightly larger and more granular than lymphocytes, a profile inconsistent with the observed phenotype of the bulk of CMKLR1+ cells. Furthermore, while circulating mouse monocytes express F4/80 and CD11 b, they do not express CD14.

To complement the surface staining data, we sorted CMKLR1+ cells and performed cytospins and Wright-Geimsa staining to examine the cellular morphology. The CMKLR1+ cells display a “fried egg” morphology: they are large, round cells with a centrally located round or reniform nucleus, and contain an abundance of cytoplasm with spec-like, blue-staining granular material. The membrane is generally ruffled with occasional protrusions. There are visible vacuoles, and pale peri-nuclear regions, perhaps indicative of Golgi apparatus. The morphology clearly indicates that the cells are not typical neutrophils, eosinophils, basophils, or lymphocytes. This result is consistent with the cell surface marker expression obtained by flow cytometry, which together indicate that CMKLR1+ are of a monocytic lineage.

We observed that CMKLR1+ cells display a selective tissue distribution. While the cells are present in peripheral blood, we were unable to detect them in spleen, lymph node, thymus or the bone marrow. In preliminary experiments, we have observed CMKLR1+ cells in peritoneal cavity lavage fluid, a site known to have large numbers of monocytic-lineage cells.

To examine the functionality of CMKLR1 expressed by mouse leukocytes, we performed in vitro transwell chemotaxis experiments using freshly isolated blood cells. We observed significant migration of large, granular F4/80+CD11b+ cells to high concentrations of recombinant human chemerin. No other major leukocyte population was observed to respond to chemerin, indicating the specificity of response.

Based on their phenotype, we predict that these cells act as macrophages, providing immune surveillance for the blood and selected tissues. Activation via pathogen associated molecular pattern binding to Toll-like receptors may stimulate the cells to engage in phagocytosis and subsequent antigen processing and presentation. CMKLR1:chemerin interactions may help recruit these cells to sites of tissue damage or inflammation, where they would differentiate and perform effector functions.

Although the foregoing invention has been described in some detail byway of illustration and example for purposes of clarity of understanding, it will be readily apparent to those of ordinary skill in the art in light of the teachings of this invention that certain changes and modifications may be made thereto without departing from the spirit or scope of the appended claims.

1. A method of modulating the trafficking of leukocytes in a mammalian host, the method comprising:
administering an effective amount of a cystatin-like chemoattractant, chemoattractant mimic, or chemoattractant inhibitor, in a dose effective to modulate said trafficking of leukocytes.

2. The method of claim 1, wherein said administration provides for a prolonged localized concentration of said cystatin-like chemoattractant agent.

3. The method of claim 1, wherein said cystatin-like chemoattractant is chemerin.

4. The method of claim 3, wherein said leukocytes are dendritic cells.

5. The method of claim 4, wherein said dendritic cells are plasmacytoid dendritic cells.

6. The method of claim 4, further comprising the step of administering a dendritic cell activating factor.

7. The method of claim 4, further comprising administering an antigen.

8. A method of increasing the immune response in a mammalian host to a immunogen, the method comprising:
administering a cystatin-like chemoattractant agonist at a target site in said host, in a dose effective to substantially increase the number of dendritic cells present at said target site;
immunizing said host with said immunogen, wherein said immunogen is introduced into said host at said target site.

9. The method of claim 8, wherein said cystatin-like chemoattractant is chemerin.

10. The method of claim 9, wherein said dendritic cells are plasmacytoid dendritic cells.

11. The method according to claim 8, wherein said target site is cutaneous.

12. The method according to claim 8, wherein said target site is intramuscular.

13. The method according to claim 8, wherein said target site is intratumor.

14. The method according to claim 8, wherein said target site is a lymph node.

15. The method according to claim 8, wherein said target site is one of Peyer's patches, spleen or thymus.

16. The method of claim 8, wherein said cystatin-like chemoattractant agonist is a peptide.

17. The method of claim 8, wherein said cystatin-like chemoattractant agonist is a non-peptide agonist.

18. The method of claim 8, wherein said immunogen and said cystatin-like chemoattractant agonist are co-formulated.

19. The method of claim 8, wherein said immunogen and said cystatin-like chemoattractant agonist are separately formulated.

20. The method according to claim 19, wherein said cystatin-like chemoattractant agonist is administered prior to said immunogen.

21. The method of claim 8, wherein said immunogen is a tumor antigen.

22. The method of claim 8, wherein said immunogen is a bacterial antigen.

23. The method of claim 8, wherein said immunogen is a viral antigen.

24. The method of claim 8, wherein said immunogen is a polypeptide.

25. The method according to claim 8, wherein said immunogen is a nucleic acid encoding a polypeptide.

26. The method according to claim 8, wherein said mammalian host is a human.

27. The method of claim 1, wherein said cystatin-like chemoattractant is selected from the group consisting of:
Cst1 (Genbank accession number NM_{—}001898); Cst2 (Genbank accession number NM_{—}001322); Cst3 (Genbank accession number NM_{—}000099); Cst4 (Genbank accession number NM_{—}001899); Cst5 (Genbank accession number NM_{—}001900); Cst6 (Genbank accession number NM_{—}001323); Cst7 (Genbank accession number NM_{—}003650); Cst8 (Genbank accession number NM_{—}005492); Cst9-like (Genbank accession number NM_{—}080610); Cst11 variant 1 (Genbank accession number NM_{—}130794); Cst11 variant 2 (Genbank accession number NM_{—}08030); CstA (or stefin A) (Genbank accession number BC010379); CstB (or stefin B) (Genbank accession number BT007040); Kininogen (Genbank accession number NM_{—}000893); Fetuin B (Genbank accession number NM_{—}014375); Cathepsin F (Genbank accession number AF088886); Invariant chain (Ii) (Genbank accession number NP_{—}004346); gamma-glutamyltransferase-like activity 3 (GGTLA3) (Genbank accession number XM_{—}066189); and histidine-rich glycoprotein (Genbank accession number M13149).

28. The method according to claim 1, comprising administering an effective amount of a cystatin-like chemoattractantantagonist, in a dose effective to reduce an immune response.
